(19) United States
(12) Patent Application Publication
(10) Pub. No.: US 2023/0339874 A1
(43) Pub. Date: Oct. 26, 2023

(54) PIPERAZINYL COMPOUNDS AND METHODS FOR TREATING NEMATODE INFECTIONS

(71) Applicant: The Governing Council of the University of Toronto, Toronto (CA)

(72) Inventors: Peter Roy, Toronto (CA); Sean Harrington, Toronto (CA); Jacob Pyche, Peterborough (CA); Andrew Burns, Toronto (CA); Rachel Baker, Toronto (CA); Mark Lautens, Toronto (CA)

(21) Appl. No.: 18/025,665

(22) PCT Filed: Sep. 10, 2021
(86) PCT No.: PCT/CA2021/051257

§ 371 Date: Mar. 10, 2023

Related U.S. Application Data
(60) Provisional application No. 63/077,149, filed on Sep. 11, 2020.

(51) Int. Cl. C07D 295/073; A01N 43/60; C07D 213/74; A61P 33/10; A01P 5/00

(52) U.S. Cl. CPC C07D 295/073; A01N 43/60; C07D 213/74; A61P 33/10; A01P 5/00

(57) ABSTRACT

The present application relates to the treatment of nematode infections. For example, the application relates to the use of compounds of Formula (I), Formula (II), Formula (III), Formula (IV), Formula (V) and/or Formula (VI) as defined herein for treatment of a nematode infection or a disease, disorder or condition arising from a nematode infection:

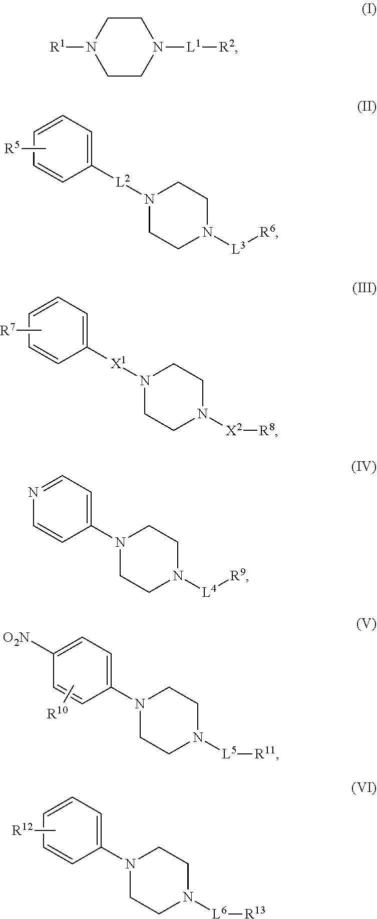

### CROSS-REFERENCE TO RELATED APPLICATIONS

The present application claims the benefit of priority from co-pending U.S. provisional patent application No. 63/077,149 filed on Sep. 11, 2020, the contents of which are incorporated herein by reference in their entirety

### FIELD

The present application relates to the treatment of nematode infections. For example, the application relates to methods and uses of compounds of Formula (I), Formula (II), Formula (III), Formula (IV), Formula (V), and/or Formula (VI) as defined herein for treatment of a nematode infection or a disease, disorder or condition arising from a nematode infection.

### INTRODUCTION

The burden of parasitic nematodes on humanity is severe. The WHO estimates that over two billion people are infected with at least one parasitic nematode species. Chronic infection can cause dietary deficiency, anemia, developmental delay, elephantiasis, blindness, and death. Human infection in the west is increasing, coincident with a warming climate and the movement of sub-tropical species northward. Intestinal nematode infections alone are responsible for an estimated disease burden of 3.4 million disability-adjusted life-years. Furthermore, nematode infestation of food increases costs and contributes to malnutrition. Nematodes have evolved widespread resistance to nearly every anthelmintic (anti-worm) drug on the market. Hence, there is a dire need for the development of new compounds that kill parasitic worms.

Most anthelmintics disrupt the worm's nervous system, which ultimately allows the mammalian host to clear the infection. For example, key targets of ivermectin are glutamate-gated chloride channels of the nervous system. Ivermectin agonizes these channels and hyperpolarizes the cell, in turn leading to paralysis. Similarly, levamisole agonizes nicotinic acetylcholine receptors leading to depolarization of muscles and, in turn, paralysis.

### SUMMARY

Families of small molecule compounds have been identified that incapacitate parasitic nematodes.

Accordingly, in an embodiment, the present application includes methods for treating or preventing a nematode infection and/or methods of treating or preventing a disease, disorder or condition arising from a nematode infection comprising administering an effective amount of a compound of Formula (I)

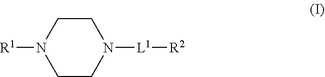

and/or a pharmaceutically acceptable salt and/or solvate thereof to a subject in need thereof
wherein
R¹ is C₁₋₄alkyl;
R² is C₃₋₈cycloalkyl or C₃₋₈cycloalkenyl, each of which is unsubstituted or substituted with one or two substituents independently selected from OH, F, Cl, Br, CN, C₁₋₄alkyl, C₁₋₄fluoroalkyl, OC₁₋₄alkyl and OC₁₋₄fluoroalkyl; and
L¹ is a direct bond or C₁₋₄alkylene.

The present application also includes methods for treating or preventing a nematode infection and/or methods of treating or preventing a disease, disorder or condition arising from a nematode infection comprising administering an effective amount of a compound of Formula (II)

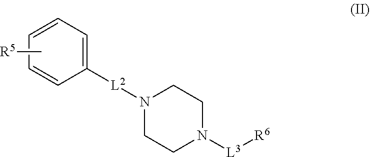

and/or a pharmaceutically acceptable salt and/or solvate thereof to a subject in need thereof
wherein:
R⁵ is C₁₋₄alkyl, C₁₋₄fluoroalkyl, NO₂, Cl, F or CN;
L² is C₁₋₄alkylene;
L³ is a direct bond or C₁₋₄alkylene; and
R⁶ is C₃₋₈cycloalkyl or C₃₋₈cycloalkenyl, each of which is unsubstituted or substituted with one or two substituents independently selected from OH, F, Cl, Br, CN, C₁₋₄alkyl, C₁₋₄fluoroalkyl, OC₁₋₄alkyl and OC₁₋₄fluoroalkyl.

The present application also includes methods for treating or preventing a nematode infection and/or methods of treating or preventing a disease, disorder or condition arising from a nematode infection comprising administering an effective amount of a compound of Formula (III)

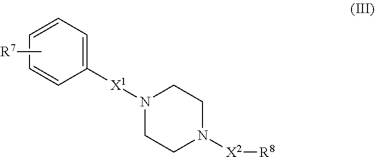

and/or a pharmaceutically acceptable salt and/or solvate thereof to a subject in need thereof
wherein:
R⁷ is H, C₁₋₄alkyl, C₁₋₄fluoroalkyl, NO₂, Cl, F or CN; one of X¹ or X² is C═O and the other is a direct bond; and
R⁸ is phenyl, C₃₋₈cycloalkyl or C₃₋₈cycloalkenyl, each of which is unsubstituted or substituted with one or two substituents independently selected from OH, F, Cl, Br, CN, C₁₋₄alkyl, C₁₋₄fluoroalkyl, OC₁₋₄alkyl and OC₁₋₄fluoroalkyl.

The present application also includes methods for treating or preventing a nematode infection and/or methods of treating or preventing a disease, disorder or condition arising from a nematode infection comprising administering an effective amount of a compound of Formula (IV)

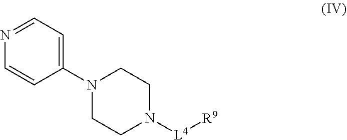

and/or a pharmaceutically acceptable salt and/or solvate thereof to a subject in need thereof
wherein:
L⁴ is a direct bond or C₁₋₄alkylene; and
R⁹ is C₃₋₈cycloalkyl or C₃₋₈cycloalkenyl, each of which is unsubstituted or substituted with one or two substituents independently selected from OH, F, Cl, Br, CN, C₁₋₄alkyl, C₁₋₄fluoroalkyl, OC₁₋₄alkyl and OC₁₋₄fluoroalkyl

The present application also includes methods for treating or preventing a nematode infection and/or methods of treating or preventing a disease, disorder or condition arising from a nematode infection comprising administering an effective amount of a compound of Formula (V)

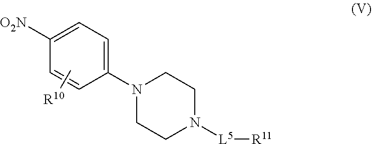

and/or a pharmaceutically acceptable salt and/or solvate thereof to a subject in need thereof wherein:
R¹⁰ is C₁₋₄alkyl, C₁₋₄fluoroalkyl, Cl, F or CN;
L⁵ is a direct bond or C₁₋₄alkylene; and
R¹¹ is C₃₋₈cycloalkyl or C₃₋₈cycloalkenyl, each of which is unsubstituted or substituted with one or two substituents independently selected from OH, F, Cl, Br, CN, C₁₋₄alkyl, C₁₋₄fluoroalkyl, OC₁₋₄alkyl and OC₁₋₄fluoroalkyl.

The present application also includes methods for treating or preventing a nematode infection and/or methods of treating or preventing a disease, disorder or condition arising from a nematode infection comprising administering an effective amount of a compound of Formula (VI)

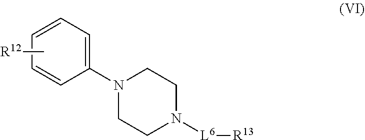

and/or a pharmaceutically acceptable salt and/or solvate thereof to a subject in need thereof
wherein:
R¹² is C₁₋₄alkyl, C₁₋₄fluoroalkyl, NO₂, Cl, F or CN;
L⁶ is a direct bond or C₁₋₄alkylene; and
R¹³ is C₃₋₈cycloalkyl or C₃₋₈cycloalkenyl, each of which is unsubstituted or substituted with one or two substituents independently selected from OH, F, Cl, Br, CN, C₁₋₄alkyl, C₁₋₄fluoroalkyl, OC₁₋₄alkyl, OC₁₋₄fluoroalkyl, and OC₁₋₄fluoroalkyl, provided that when R¹² is NO₂ or CN and is in a position para to the piperazine on the phenyl ring, then R¹³ is not C₃₋₈cycloalkyl or C₃₋₈cycloalkenyl each of which is unsubstituted, or C₃₋₈cycloalkyl or C₃₋₈cycloalkenyl each of which is substituted with one or two substituents independently selected from C₁₋₄alkyl and C₁₋₄fluoroalkyl.

In an embodiment, the present application includes pharmaceutical compositions comprising a pharmaceutically acceptable carrier and a compound of Formula (I), Formula (II), Formula (III), Formula (IV), Formula (V), and/or Formula (VI), wherein the compound of Formula (I), Formula (II), Formula (III), Formula (IV), Formula (V), and/or Formula (VI), is present in an amount effective to treat a nematode infection in a subject in need thereof.

In an embodiment, the present application includes uses of pharmaceutical compositions comprising a compound of Formula (I), Formula (II), Formula (III), Formula (IV), Formula (V), and/or Formula (VI) for treating or preventing a nematode infection or a disease, a disorder, or a condition arising from a nematode infection in a subject in need thereof.

In an embodiment, the present application includes nematicidal compositions comprising a carrier and a compound of Formula (I), Formula (II), Formula (III), Formula (IV), Formula (V), and/or Formula (VI) wherein the compound of Formula (I), Formula (II), Formula (III), Formula (IV), Formula (V), and/or Formula (VI) is present in an amount effective to treat a nematode infection in a subject in need thereof.

In an embodiment, the present application includes uses of the nematicidal composition described herein for treating or preventing a nematode infection or a disease, a disorder, or a condition arising from a nematode infection in a subject in need thereof.

In an embodiment, the present application includes methods of treating or preventing a nematode infection comprising administering an effective amount of a nematicidal composition described herein to a subject in need thereof.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### DESCRIPTION OF VARIOUS EMBODIMENTS

### I. Definitions

Unless otherwise indicated, the definitions and embodiments described in this and other sections are intended to be applicable to all embodiments and aspects of the present application herein described for which they are suitable as would be understood by a person skilled in the art.

The term “compound(s) of the application” or “compound(s) of the present application” and the like as used herein refers to a compound of Formula (I), Formula (II), Formula (III), Formula (IV), Formula (V), and/or Formula (VI) or pharmaceutically acceptable salts and/or solvates thereof.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

The term “consisting” and its derivatives, as used herein, are intended to be closed terms that specify the presence of the stated features, elements, components, groups, integers, and/or steps, but exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

As used in this application, the singular forms “a”, “an” and “the” include plural references unless the content clearly dictates otherwise. For example, an embodiment including “a compound” should be understood to present certain aspects with compound or two or more additional compounds.

In embodiments comprising an “additional” or “second” component, such as an additional or second compound, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present. The term “and/or” with respect to pharmaceutically acceptable, salts and/or solvates thereof means that referenced compounds exist as individual salts or hydrates, as well as a combination of, for example, a salt of a solvate of a compound.

The term “suitable” as used herein means that the selection of the particular compound or conditions would depend on the specific synthetic manipulation to be performed, the identity of the molecule(s) to be transformed and/or the specific use for the compound, but the selection would be well within the skill of a person trained in the art. All process/method steps described herein are to be conducted under conditions sufficient to provide the product shown. A person skilled in the artwould understand that all reaction conditions, including, for example, reaction solvent, reaction time, reaction temperature, reaction pressure, reactant ratio and whether or not the reaction should be performed under an anhydrous or inert atmosphere, can be varied to optimize the yield of the desired product and it is within their skill to do so.

The present description refers to a number of chemical terms and abbreviations used by those skilled in the art. Nevertheless, definitions of selected terms are provided for clarity and consistency.

The term “alkyl” as used herein, whether it is used alone or as part of another group, means straight or branched chain, saturated alkyl groups. The number of carbon atoms that are possible in the referenced alkyl group are indicated by the prefix “Cₙ₁₋ₙ₂”. For example, the term C₁₋₁₀alkyl means an alkyl group having 1, 2, 3, 4, 5, 6, 7, 8, 9 or 10 carbon atoms.

The term “alkylene”, whether it is used alone or as part of another group, means straight or branched chain, saturated alkylene group, that is, a saturated carbon chain that contains substituents on two of its ends. The number of carbon atoms that are possible in the referenced alkylene group are indicated by the prefix “Cₙ₁₋ₙ₂”. For example, the term C₂₋₆alkylene means an alkylene group having 2, 3, 4, 5 or 6 carbon atoms.

The term “cycloalkyl,” as used herein, whether it is used alone or as part of another group, means a saturated carbocyclic group containing one or more rings. The number of carbon atoms that are possible in the referenced cycloalkyl group are indicated by the numerical prefix “Cₙ₁₋ₙ₂”. For example, the term C₃₋₈cycloalkyl means a cycloalkyl group having 3, 4, 5, 6, 7, or 8 carbon atoms.

The term “cycloalkenyl” as used herein, whether it is used alone or as part of another group, means cyclic, unsaturated alkyl groups. For example, the term C₃₋₈cycloalkenyl means a cycloalkenyl group having 3, 4, 5, 6, 7, or 8 carbon atoms and at least one double bond.

All cyclic groups, including phenyl, cycloalkyl and cycloalkenyl groups, contain one or more than one ring (i.e. are polycyclic). When a cyclic group contains more than one ring, the rings may be fused, bridged, spirofused or linked by a bond.

The term “benzofused” as used herein refers to a polycyclic group in which a benzene ring is fused with another ring.

A first ring being “fused” with a second ring means the first ring and the second ring share two adjacent atoms there between.

A first ring being “bridged” with a second ring means the first ring and the second ring share two non-adjacent atoms there between.

A first ring being “spirofused” with a second ring means the first ring and the second ring share one atom there between.

The term “substituted” as used herein means that the referenced atom contains at least one substituent group other that a hydrogen atom.

The term “nematode infection” as used herein refers to an invasion of cells or bodily tissues by a foreign undesirable nematode.

The term “anthelmintic” or “anthelmintics” as used herein refers to a group of antiparasitic drug used in the treatment and prevention of nematode infections.

As used herein, a compound with “anthelmintic activity” is a compound, which when tested, has measurable nematode-killing activity or results in sterility or reduced fertility in the nematodes such that fewer viable or no offspring result, or compromises the ability of the nematode to infect or reproduce in its host, or interferes with the growth or development of a nematode. The compound may also display nematode repellant properties.

The term “pharmaceutically acceptable” means compatible with the treatment of subjects.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to a subject.

The term “pharmaceutically acceptable salt” means an acid addition salt or a basic addition salt suitable for, or compatible with, the treatment of subjects.

An acid addition salt suitable for, or compatible with, the treatment of subjects is any non-toxic organic or inorganic acid addition salt of any basic compound.

A base addition salt suitable for, or compatible with, the treatment of subjects is any non-toxic organic or inorganic base addition salt of any acidic compound.

The term “solvates” as used herein refers to complexes formed between a compound and a solvent from which the compound is precipitated or in which the compound is made. Accordingly, the term “solvate” as used herein means a compound, or a salt a compound, wherein molecules of a suitable solvent are incorporated in the crystal lattice.

The term “pharmaceutically acceptable solvate” means a solvate suitable for, or compatible with, the treatment of subjects. For pharmaceutically acceptable solvates, a suitable solvent is physiologically tolerable at the dosage used or administered.

The expression “disease, disorder or condition arising from a nematode infection” as used herein refers to any disease, disorder or condition that is directly or indirectly caused by the presence of a nematode infection in a subject.

The term “subject” as used herein includes plants, seeds, soil, and all members of the animal kingdom including mammals and birds, and their food. Thus, the methods of the present application are applicable to plant treatment, human therapy and veterinary applications.

When used, for example, with respect to the methods of treatment, uses, compositions and kits of the application, a subject, for example a subject “in need thereof” is a subject who has been diagnosed with, is suspected of having, may come in to contact with, and/or was previously treated for a nematode infection or a disease, disorder or condition arising from a nematode infection.

When used, for example, in respect to plant treatments, the compounds and/or compositions may be delivered by several means including pre-planting, post-planting and as a feed additive, drench, external application, pill or by injection.

The term “pharmaceutical composition” as used herein refers to a composition of matter for pharmaceutical use.

The term “pharmaceutically acceptable” means compatible with the treatment of subjects.

The term “nematicidal composition” as used here in refers to a composition of matter for managing one or more nematode infections.

The term “administered” as used herein means administration of an effective amount of a compound, including compounds of Formula I or II, or a salt and/or solvate thereof, to a cell either in cell culture or in a subject.

As used herein, the term “effective amount” or “therapeutically effective amount” means an amount effective, at dosages and for periods of time necessary to achieve a desired result. For example, in the context of treating a nematode infection, or a disease, disorder or condition arising from a nematode infection, an effective amount of a compound is an amount that, for example, reduces the nematode infection compared to the nematode infection without administration of the compound. By “reducing the infection”, it is meant, for example, reducing the amount of the infectious agent in the subject and/or reducing the symptoms of the infection. The amount of a given compound or composition that will correspond to such an amount will vary depending upon various factors, such as the given compound or composition, the formulation, the route of administration, the type of condition, disease or disorder, the identity of the subject being treated, and the like, but can nevertheless be routinely determined by one skilled in the art.

The terms “to treat”, “treating” and “treatment” as used herein and as is well understood in the art, means an approach for obtaining beneficial or desired results, including clinical results. Beneficial or desired clinical results include, but are not limited to, diminishment of extent of nematode infection, stabilization (i.e. not worsening) of the state of the nematode infection, preventing spread of the nematode infection, delay or slowing of infection progression, amelioration or palliation of the nematode infectious state, diminishment of the reoccurrence of nematode infection, diminishment, stabilization, alleviation or amelioration of one or more diseases, disorders or conditions arising from the nematode infection, diminishment of the reoccurrence of one or more diseases, disorders or conditions arising from the nematode infection, and remission of the nematode infection and/or one or more symptoms or conditions arising from the nematode infection, whether partial or total, whether detectable or undetectable. “To treat”, “treating” and “treatment” can also mean prolonging survival as compared to expected survival if not receiving treatment. “To treat”, “treating” and “treatment” as used herein also include prophylactic treatment. For example, a subject with an early nematode infection is treated to prevent progression, or alternatively a subject in remission is treated to prevent recurrence.

“Palliating” an infection, disease, disorder and/or condition means that the extent and/or undesirable manifestations of an infection, disease, disorder and/or condition are lessened and/or time course of the progression is slowed or lengthened, as compared to not treating the infection, disease, disorder and/or condition.

The term “prevention” or “prophylaxis” and the like as used herein refers to a reduction in the risk or probability of a subject becoming afflicted with a nematode infection and/or a disease, disorder and/or condition arising from a nematode infection or manifesting a symptom associated with a nematode infection and/or a disease, disorder and/or condition arising from a nematode infection.

### II. Methods and Uses of the Application

Families of small molecule compounds have been identified that incapacitate parasitic nematodes.

Accordingly, the present application includes methods for treating or preventing a nematode infection comprising administering an effective amount of a compound of Formula (I)

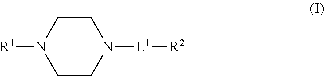

and/or a pharmaceutically acceptable salt and/or solvate thereof to a subject in need thereof
wherein
R¹ is C₁₋₄alkyl;
R² is C₃₋₈cycloalkyl or C₃₋₈cycloalkenyl, each of which is unsubstituted or substituted with one or two substituents independently selected from OH, F, Cl, Br, CN, C₁₋₄alkyl, C₁₋₄fluoroalkyl, OC₁₋₄alkyl and OC₁₋₄fluoroalkyl; and
L¹ is a direct bond or C₁₋₄alkylene.

The application also includes a use of one or more compounds of Formula (I) for treating or preventing a nematode infection as well as a use of one or more compounds of Formula (I) for preparing a medicament for treating or preventing a nematode infection. The application further includes one or more compounds of Formula (I) for use to treat or prevent a nematode infection.

In an embodiment, the present application includes methods of treating or preventing a disease, disorder or condition arising from a nematode infection comprising administering an effective amount a compound of Formula (I)

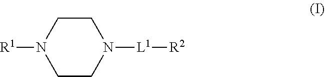

and/or a pharmaceutically acceptable salt and/or solvate thereof to a subject in need thereof
wherein
R¹ is C₁₋₄alkyl;
R² is C₃₋₈cycloalkyl or C₃₋₈cycloalkenyl, each of which is unsubstituted or substituted with one or two substituents independently selected from OH, F, Cl, Br, CN, C₁₋₄alkyl, C₁₋₄fluoroalkyl, OC₁₋₄alkyl and OC₁₋₄fluoroalkyl; and
L¹ is a direct bond or C₁₋₄alkylene.

The application also includes a use of one or more compounds of Formula (I) for treating or preventing a disease, disorder or condition arising from a nematode infection as well as a use of one or more compounds of Formula (I) for preparing a medicament for treating or preventing a disease, disorder or condition arising from a nematode infection. The application further includes one or more compounds of Formula (I) for use to treat or prevent a disease, disorder or condition arising from a nematode infection.

In an embodiment, R¹ is C₁₋₃alkyl. In an embodiment, R¹ is methyl, ethyl, propyl or isopropyl. In an embodiment, R¹ is methyl or ethyl.

In an embodiment, R² is C₃₋₈cycloalkenyl. In an embodiment, the C₃₋₈ cycloalkenyl in R² is cyclopentenyl or cyclohexenyl. In an embodiment, the C₃₋₈ cycloalkenyl in R² is cyclohexenyl. In an embodiment, the C₃₋₈cycloalkenyl in R² is a bicyclic cycloalkenyl. In an embodiment, the bicyclic cycloalkenyl is bicycloheptenyl or bicyclooctenyl. In an embodiment, the bicyclic cycloalkenyl is bicycloheptenyl. In an embodiment, R² is C₃₋₈cycloalkyl. In an embodiment, the C₃₋₈cycloalkyl in R² is cyclobutyl, cyclopentyl or cyclohexyl. In an embodiment, the C₃₋₈cycloalkyl in R² is cyclohexyl. In an embodiment, the C₃₋₈cycloalkyl in R² is a bicyclic cycloalkyl. In an embodiment, the bicyclic cycloalkyl is bicycloheptyl or bicyclooctyl. In an embodiment, the bicyclic cycloalkyl is bicycloheptyl.

In an embodiment, R² is C₃₋₈cycloalkyl or C₃₋₈cycloalkenyl each of which is unsubstituted. In an embodiment, R² is C₃₋₈cycloalkyl or C₃₋₈cycloalkenyl each of which is substituted with one or two substituents independently selected from OH, F, Cl, Br, CN, C₁₋₄alkyl, C₁₋₄fluoroalkyl, OC₁₋₄alkyl and OC₁₋₄fluoroalkyl. In an embodiment, R² is C₃₋₈ cycloalkyl or C₃₋₈cycloalkenyl, each of which substituted with one or two substituents independently selected from OH, F, Br, Cl, CN, C₁₋₂alkyl, C₁₋₂fluoroalkyl, OC₁₋₂alkyl and OC₁₋₂fluoroalkyl. In an embodiment, R² is C₃₋₈cycloalkyl or C₃₋₈cycloalkenyl, each of which substituted with one or two substituents independently selected from OH, F, Br, Cl, CN, CH₃, CH₂CH₃, CF₃, OCH₃, and OCF₃.

In an embodiment, L¹ is a direct bond. In an embodiment, L¹ is C₁₋₄alkylene. In an embodiment, L¹ is C₁₋₃alkylene. In an embodiment, L¹ is C₁alkylene or C₂alkylene. In an embodiment, L¹ is C₁alkylene.

In an embodiment, the compound of Formula (I) is

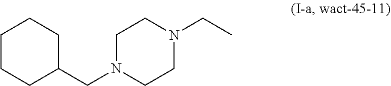

and/or a pharmaceutically acceptable salt and/or solvate thereof.

In an embodiment, the present application includes methods for treating or preventing a nematode infection comprising administering an effective amount of a compound of Formula (II)

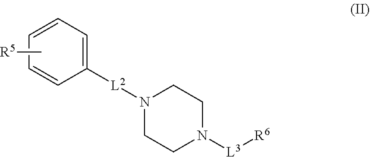

and/or a pharmaceutically acceptable salt and/or solvate thereof to a subject in need thereof
wherein:
R⁵ is C₁₋₄alkyl, C₁₋₄fluoroalkyl, NO₂, Cl, F or CN;
L² is C₁₋₄alkylene;
L³ is a direct bond or C₁₋₄alkylene; and
R⁶ is C₃₋₈cycloalkyl or C₃₋₈cycloalkenyl, each of which is unsubstituted or substituted with one or two substituents independently selected from OH, F, Cl, Br, CN, C₁₋₄alkyl, C₁₋₄fluoroalkyl, OC₁₋₄alkyl and OC₁₋₄fluoroalkyl.

The application also includes a use of one or more compounds of Formula (II) for treating or preventing a nematode infection as well as a use of one or more compounds of Formula (III) for preparing a medicament for treating or preventing a nematode infection. The application further includes one or more compounds of Formula (II) for use to treat or prevent a nematode infection.

In an embodiment, the present application includes methods of treating or preventing a disease, disorder or condition arising from a nematode infection comprising administering an effective amount a compound of Formula (II)

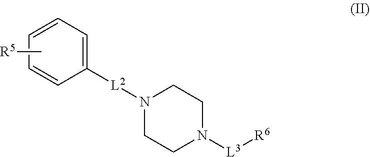

and/or a pharmaceutically acceptable salt and/or solvate thereof to a subject in need thereof
wherein:
R⁵ is C₁₋₄alkyl, C₁₋₄fluoroalkyl, NO₂, Cl, F or CN;
L² is C₁₋₄alkylene;
L³ is a direct bond or C₁₋₄alkylene; and
R⁶ is C₃₋₈cycloalkyl or C₃₋₈cycloalkenyl, each of which is unsubstituted or substituted with one or two substituents independently selected from OH, F, Cl, Br, CN, C₁₋₄alkyl, C₁₋₄fluoroalkyl, OC₁₋₄alkyl and OC₁₋₄fluoroalkyl.

The application also includes a use of one or more compounds of Formula (III) for treating or preventing a disease, disorder or condition arising from a nematode infection as well as a use of one or more compounds of Formula (II) for preparing a medicament for treating or preventing a disease, disorder or condition arising from a nematode infection. The application further includes one or more compounds of Formula (II) for use to treat or prevent a disease, disorder or condition arising from a nematode infection.

In an embodiment, R⁵ is C₁₋₂alkyl, C₁₋₂fluoroalkyl, NO₂, Cl, F or CN. In an embodiment, R⁵ is CH₃, CH₂CH₃, CF₃, NO₂, Cl, F or CN. In an embodiment, R⁵ is CH₃, CH₂CH₃, or CF₃. In an embodiment, R⁵ is NO₂, F or CN. In an embodiment, R⁵ is NO₂. In an embodiment, R⁵ is NO₂ and is in a position para to the piperazine on the phenyl ring. In an embodiment, R⁵ is NO₂ and is in a position meta to the piperazine on the phenyl ring.

In an embodiment, L² is C₁₋₃alkylene. In an embodiment, L² is C₁alkylene or C₂alkylene. In an embodiment, L² is C₁alkylene (CH₂).

In an embodiment, L³ is a direct bond. In an embodiment, L³ is C₁₋₄alkylene. In an embodiment, L³ is C₁₋₃alkylene. In an embodiment, L³ is C₁alkylene or C₂alkylene. In an embodiment, L³ is C₁alkylene (CH₂).

In an embodiment, R⁶ is C₃₋₈cycloalkenyl. In an embodiment, the C₃₋₈cycloalkenyl in R⁶ is cyclopentenyl or cyclohexenyl. In an embodiment, the C₃₋₈cycloalkenyl in R⁶ is a bicyclic cycloalkenyl. In an embodiment, the bicyclic cycloalkenyl is bicycloheptenyl or bicyclooctenyl. In an embodiment, the bicyclic cycloalkenyl is bicycloheptenyl. In an embodiment, R⁶ is C₃₋₈cycloalkyl. In an embodiment, the C₃₋₈cycloalkyl in R⁶ is cyclobutyl, cyclopentyl or cyclohexyl. In an embodiment, the C₃₋₈cycloalkyl in R⁶ is cyclopentyl or cyclohexyl. In an embodiment, the C₃₋₈cycloalkyl in R⁶ is a bicyclic cycloalkyl. In an embodiment, the bicyclic cycloalkyl is bicycloheptyl or bicyclooctyl. In an embodiment, the bicyclic cycloalkyl is bicycloheptyl.

In an embodiment, R⁶ is C₃₋₈cycloalkyl or C₃₋₈cycloalkenyl each of which is unsubstituted. In an embodiment, R⁶ is C₃₋₈cycloalkyl or C₃₋₈cycloalkenyl each of which is substituted with one or two substituents independently selected from OH, F, Cl, Br, CN, C₁₋₄alkyl, C₁₋₄fluoroalkyl, OC₁₋₄alkyl and OC₁₋₄fluoroalkyl. In an embodiment, R⁶ is C₃₋₈cycloalkyl or C₃₋₈cycloalkenyl, each of which substituted with one or two substituents independently selected from OH, F, Br, Cl, CN, C₁₋₂alkyl, C₁₋₂fluoroalkyl, OC₁₋₂alkyl and OC₁₋₂fluoroalkyl. In an embodiment, R⁶ is C₃₋₈cycloalkyl or C₃₋₈cycloalkenyl, each of which substituted with one or two substituents independently selected from OH, F, Br, Cl, CN, CH₃, CH₂CH₃, CF₃, OCH₃, and OCF₃.

In an embodiment, the compound of Formula (II) is selected from

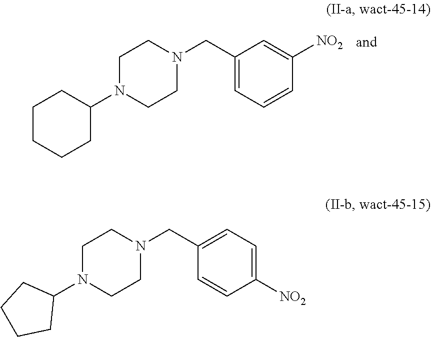 [SMILES: O=[N+]([O-])c1cccc(CN2CCN(C3CCCCC3)CC2)c1 ; O=[N+]([O-])c1ccc(CN2CCN(C3CCCC3)CC2)cc1]

and/or a pharmaceutically acceptable salt and/or solvate thereof.

In an embodiment, the present application includes methods for treating or preventing a nematode infection comprising administering an effective amount of a compound of Formula (III)

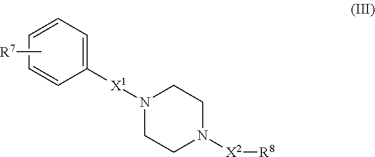

and/or a pharmaceutically acceptable salt and/or solvate thereof to a subject in need thereof
wherein:
R⁷ is H, C₁₋₄alkyl, C₁₋₄fluoroalkyl, NO₂, Cl, F or CN; one of X¹ or X² is C═O and the other is a direct bond; and
R⁸ is phenyl, C₃₋₈cycloalkyl or C₃₋₈cycloalkenyl, each of which is unsubstituted or substituted with one or two substituents independently selected from OH, F, Cl, Br, CN, C₁₋₄alkyl, C₁₋₄fluoroalkyl, OC₁₋₄alkyl and OC₁₋₄fluoroalkyl.

The application also includes a use of one or more compounds of Formula (III) for treating or preventing a nematode infection as well as a use of one or more compounds of Formula (IV) for preparing a medicament for treating or preventing a nematode infection. The application further includes one or more compounds of Formula (III) for use to treat or prevent a nematode infection.

In an embodiment, the present application includes methods of treating or preventing a disease, disorder or condition arising from a nematode infection comprising administering an effective amount a compound of Formula (III)

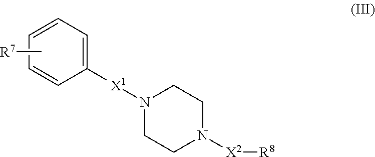

and/or a pharmaceutically acceptable salt and/or solvate thereof to a subject in need thereof
wherein:
R⁷ is H, C₁₋₄alkyl, C₁₋₄fluoroalkyl, NO₂, Cl, F or CN; one of X¹ or X² is C═O and the other is a direct bond; and
R⁸ is phenyl, C₃₋₈cycloalkyl or C₃₋₈cycloalkenyl, each of which is unsubstituted or substituted with one or two substituents independently selected from OH, F, Cl, Br, CN, C₁₋₄alkyl, C₁₋₄fluoroalkyl, OC₁₋₄alkyl and OC₁₋₄fluoroalkyl.

The application also includes a use of one or more compounds of Formula (III) for treating or preventing a disease, disorder or condition arising from a nematode infection as well as a use of one or more compounds of Formula (III) for preparing a medicament for treating or preventing a disease, disorder or condition arising from a nematode infection. The application further includes one or more compounds of Formula (III) for use to treat or prevent a disease, disorder or condition arising from a nematode infection.

In an embodiment, R⁷ is H, C₁₋₂alkyl, C₁₋₂fluoroalkyl, NO₂, Cl, F or CN. In an embodiment, R⁷ is H, CH₃, CH₂CH₃, CF₃, NO₂, Cl, F or CN. In an embodiment, R⁷ is H, CH₃, CH₂CH₃, or CF₃. In an embodiment, R⁷ is H. In an embodiment, R⁷ is CH₃, CH₂CH₃, or CF₃. In an embodiment, R⁷ is NO₂, F or CN. In an embodiment, R⁷ is NO₂ or F.

In an embodiment, X¹ is C═O and X² is a direct bond. In an embodiment, X¹ is a direct bond and X² is C═O.

In an embodiment, R⁸ is phenyl. In an embodiment, R⁸ is C₃₋₈cycloalkenyl. In an embodiment, the C₃₋₈cycloalkenyl in R⁸ is cyclopentenyl or cyclohexenyl. In an embodiment, the C₃₋₈cycloalkenyl in R⁸ is a bicyclic cycloalkenyl. In an embodiment, the bicyclic cycloalkenyl is bicycloheptenyl or bicyclooctenyl. In an embodiment, the bicyclic cycloalkenyl is bicycloheptenyl. In an embodiment, R⁸ is C₃₋₈cycloalkyl. In an embodiment, the C₃₋₈cycloalkyl in R⁸ is cyclobutyl, cyclopentyl or cyclohexyl. In an embodiment, the C₃₋₈ cycloalkyl in R⁸ is a bicyclic cycloalkyl. In an embodiment, the bicyclic cycloalkyl is bicycloheptyl or bicyclooctyl. In an embodiment, the bicyclic cycloalkyl is bicycloheptyl.

In an embodiment, R⁸ is phenyl, C₃₋₈cycloalkyl or C₃₋₈cycloalkenyl, each of which is unsubstituted. In an embodiment, R⁸ is phenyl, C₃₋₈cycloalkyl or C₃₋₈cycloalkenyl, each of which is substituted with one or two substituents independently selected from OH, F, Cl, Br, CN, C₁₋₄alkyl, C₁₋₄fluoroalkyl, OC₁₋₄alkyl and OC₁₋₄fluoroalkyl. In an embodiment, R⁸ is phenyl, C₃₋₈cycloalkyl or C₃₋₈cycloalkenyl, each of which substituted with one or two substituents from OH, F, Br, Cl, CN, C₁₋₂alkyl, C₁₋₂fluoroalkyl, OC₁₋₂alkyl and OC₁₋₂fluoroalkyl. In an embodiment, R⁸ is phenyl, C₃₋₈cycloalkyl or C₃₋₈cycloalkenyl, each of which substituted with one or two substituents selected from OH, F, Br, Cl, CN, CH₃, CH₂CH₃, CF₃, OCH₃, and OCF₃. In an embodiment, R⁸ is phenyl which substituted with one or two substituents independently selected from OH, F, Br, Cl, and CF₃. In an embodiment, R⁸ is phenyl which substituted Br.

In an embodiment, the compound of Formula (III) is selected from

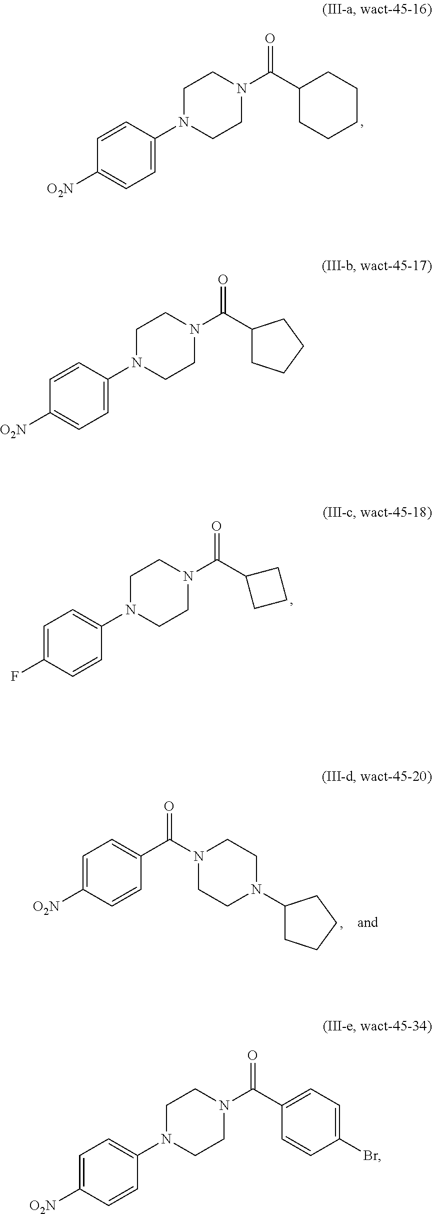 [SMILES: O=C(C1CCCCC1)N1CCN(c2ccc([N+](=O)[O-])cc2)CC1 ; O=C(C1CCCC1)N1CCN(c2ccc([N+](=O)[O-])cc2)CC1 ; O=C(C1CCC1)N1CCN(c2ccc(F)cc2)CC1 ; O=C(c1ccc([N+](=O)[O-])cc1)N1CCN(C2CCCC2)CC1 ; O=C(c1ccc(Br)cc1)N1CCN(c2ccc([N+](=O)[O-])cc2)CC1]

and/or a pharmaceutically acceptable salt and/or solvate thereof.

In an embodiment, the present application includes methods for treating or preventing a nematode infection comprising administering an effective amount of a compound of Formula (IV)

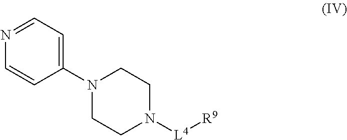

and/or a pharmaceutically acceptable salt and/or solvate thereof to a subject in need thereof
wherein:
L⁴ is a direct bond or C₁₋₄alkylene; and
R⁹ is C₃₋₈cycloalkyl or C₃₋₈cycloalkenyl, each of which is unsubstituted or substituted with one or two substituents independently selected from OH, F, Cl, Br, CN, C₁₋₄alkyl, C₁₋₄fluoroalkyl, OC₁₋₄alkyl and OC₁₋₄fluoroalkyl.

The application also includes a use of one or more compounds of Formula (IV) for treating or preventing a nematode infection as well as a use of one or more compounds of Formula (IV) for preparing a medicament for treating or preventing a nematode infection. The application further includes one or more compounds of Formula (IV) for use to treat or prevent a nematode infection.

In an embodiment, the present application includes methods of treating or preventing a disease, disorder or condition arising from a nematode infection comprising administering an effective amount a compound of Formula (IV)

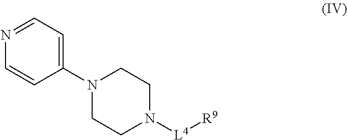

and/or a pharmaceutically acceptable salt and/or solvate thereof to a subject in need thereof
wherein:
L⁴ is a direct bond or C₁₋₄alkylene; and
R⁹ is C₃₋₈cycloalkyl or C₃₋₈cycloalkenyl, each of which is unsubstituted or substituted with one or two substituents independently selected from OH, F, Cl, Br, CN, C₁₋₄alkyl, C₁₋₄fluoroalkyl, OC₁₋₄alkyl and OC₁₋₄fluoroalkyl.

The application also includes a use of one or more compounds of Formula (IV) for treating or preventing a disease, disorder or condition arising from a nematode infection as well as a use of one or more compounds of Formula (IV) for preparing a medicament for treating or preventing a disease, disorder or condition arising from a nematode infection. The application further includes one or more compounds of Formula (IV) for use to treat or prevent a disease, disorder or condition arising from a nematode infection.

In an embodiment, L⁴ is a direct bond. In an embodiment, L⁴ is C₁₋₄alkylene. In an embodiment, L⁴ is C₁₋₃alkylene. In an embodiment, L⁴ is C₁alkylene or C₂alkylene. In an embodiment, L⁴ is C₁alkylene (CH₂).

In an embodiment, R⁹ is C₃₋₈cycloalkenyl. In an embodiment, the C₃₋₈cycloalkenyl in R⁹ is cyclopentenyl or cyclohexenyl. In an embodiment, the C₃₋₈cycloalkenyl in R⁹ is a bicyclic cycloalkenyl. In an embodiment, the bicyclic cycloalkenyl is bicycloheptenyl or bicyclooctenyl. In an embodiment, the bicyclic cycloalkenyl is bicycloheptenyl. In an embodiment, R⁹ is C₃₋₈cycloalkyl. In an embodiment, the C₃₋₈cycloalkyl in R⁹ is cyclobutyl, cyclopentyl or cyclohexyl. In an embodiment, R⁹ is cyclopentyl or cyclohexyl. In an embodiment, the C₃₋₈cycloalkyl in R⁹ is a bicyclic cycloalkyl. In an embodiment, the bicyclic cycloalkyl is bicycloheptyl or bicyclooctyl. In an embodiment, the bicyclic cycloalkyl is bicycloheptyl.

In an embodiment, R⁹ is C₃₋₈cycloalkyl or C₃₋₈cycloalkenyl each of which is unsubstituted. In an embodiment, R⁹ is C₃₋₈cycloalkyl or C₃₋₈cycloalkenyl each of which is substituted with one or two substituents independently selected from OH, F, Cl, Br, CN, C₁₋₄alkyl, C₁₋₄fluoroalkyl, OC₁₋₄alkyl and OC₁₋₄fluoroalkyl. In an embodiment, R⁹ is C₃₋₈cycloalkyl or C₃₋₈cycloalkenyl, each of which substituted with one or two substituents independently selected from OH, F, Br, Cl, CN, C₁₋₂alkyl, C₁₋₂fluoroalkyl, OC₁₋₂alkyl and OC₁₋₂fluoroalkyl. In an embodiment, R⁹ is C₃₋₈cycloalkyl or C₃₋₈cycloalkenyl, each of which substituted with one or two substituents independently selected from OH, F, Br, Cl, CN, CH₃, CH₂CH₃, CF₃, OCH₃, and OCF₃.

In an embodiment, the compound of Formula (IV) is selected from

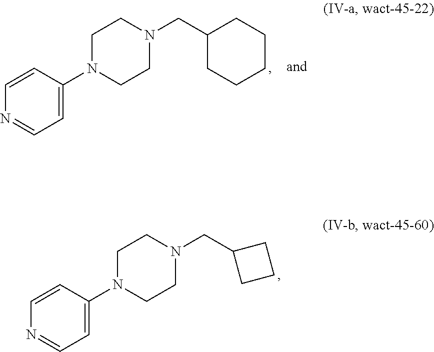 [SMILES: c1cc(N2CCN(CC3CCCCC3)CC2)ccn1 ; c1cc(N2CCN(CC3CCC3)CC2)ccn1]

and/or a pharmaceutically acceptable salt and/or solvate thereof.

In an embodiment, the present application includes methods for treating or preventing a nematode infection comprising administering an effective amount of a compound of Formula (V)

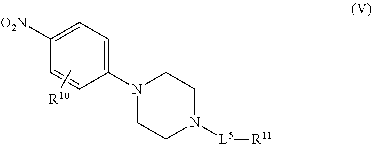

and/or a pharmaceutically acceptable salt and/or solvate thereof to a subject in need thereof
wherein:
R¹⁰ is C₁₋₄alkyl, C₁₋₄fluoroalkyl, Cl, F or CN;
L⁵ is a direct bond or C₁₋₄alkylene; and
R¹¹ is C₃₋₈cycloalkyl or C₃₋₈cycloalkenyl, each of which is unsubstituted or substituted with one or two substituents independently selected from OH, F, Cl, Br, CN, C₁₋₄alkyl, C₁₋₄fluoroalkyl, OC₁₋₄alkyl and OC₁₋₄fluoroalkyl.

The application also includes a use of one or more compounds of Formula (V) for treating or preventing a nematode infection as well as a use of one or more compounds of Formula (V) for preparing a medicament for treating or preventing a nematode infection. The application further includes one or more compounds of Formula (V) for use to treat or prevent a nematode infection.

In an embodiment, the present application includes methods of treating or preventing a disease, disorder or condition arising from a nematode infection comprising administering an effective amount a compound of Formula (V)

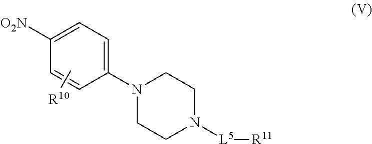

and/or a pharmaceutically acceptable salt and/or solvate thereof to a subject in need thereof,
wherein:
R¹⁰ is C₁₋₄alkyl, C₁₋₄fluoroalkyl, Cl, F or CN;
L⁵ is a direct bond or C₁₋₄alkylene; and
R¹¹ is C₃₋₈cycloalkyl or C₃₋₈cycloalkenyl, each of which is unsubstituted or substituted with one or two substituents independently selected from OH, F, Cl, Br, CN, C₁₋₄alkyl, C₁₋₄fluoroalkyl, OC₁₋₄alkyl and OC₁₋₄fluoroalkyl.

The application also includes a use of one or more compounds of Formula (V) for treating or preventing a disease, disorder or condition arising from a nematode infection as well as a use of one or more compounds of Formula (V) for preparing a medicament for treating or preventing a disease, disorder or condition arising from a nematode infection. The application further includes one or more compounds of Formula (V) for use to treat or prevent a disease, disorder or condition arising from a nematode infection.

In an embodiment, R¹⁰ is C₁₋₂alkyl, C₁₋₂fluoroalkyl, Cl, F or CN. In an embodiment, R¹⁰ is CH₃, CH₂CH₃, CF₃, Cl, F or CN. In an embodiment, R¹⁰ is CH₃, CH₂CH₃, Cl, F, CN or CF₃. In an embodiment, R¹⁰ is CH₃, CH₂CH₃ or CF₃. In an embodiment, R¹⁰ is CH₃ and is in a position meta to the piperazine on the phenyl ring and is in a position ortho to the piperazine on the phenyl ring.

In an embodiment, L⁵ is a direct bond. In an embodiment, L⁵ is C₁₋₄alkylene. In an embodiment, L⁵ is C₁₋₃alkylene. In an embodiment, L⁵ is C₁alkylene or C₂alkylene. In an embodiment, L⁵ is C₁alkylene (CH₂).

In an embodiment, R¹¹ is C₃₋₈cycloalkenyl. In an embodiment, the C₃₋₈ cycloalkenyl in R¹¹ cyclopentenyl or cyclohexenyl. In an embodiment, the C₃₋₈ cycloalkenyl in R¹¹ is a bicyclic cycloalkenyl. In an embodiment, the bicyclic cycloalkenyl is bicycloheptenyl or bicyclooctenyl. In an embodiment, the bicyclic cycloalkenyl is bicycloheptenyl. In an embodiment, R¹¹ is C₃₋₈cycloalkyl. In an embodiment, the C₃₋₈ cycloalkyl in R¹¹ is cyclobutyl, cyclopentyl or cyclohexyl. In an embodiment, the C₃₋₈ cycloalkyl in R¹¹ is cyclohexyl. In an embodiment, the C₃₋₈cycloalkyl in R¹¹ is a bicyclic cycloalkyl. In an embodiment, the bicyclic cycloalkyl is bicycloheptyl or bicyclooctyl. In an embodiment, the bicyclic cycloalkyl is bicycloheptyl.

In an embodiment, R¹¹ is C₃₋₈cycloalkyl or C₃₋₈cycloalkenyl each of which is unsubstituted. In an embodiment, R¹¹ is C₃₋₈cycloalkyl or C₃₋₈cycloalkenyl each of which is substituted with one or two substituents independently selected from OH, F, Cl, Br, CN, C₁₋₄alkyl, C₁₋₄fluoroalkyl, OC₁₋₄alkyl and OC₁₋₄fluoroalkyl. In an embodiment, R⁹ is C₃₋₈cycloalkyl or C₃₋₈cycloalkenyl, each of which substituted with one or two substituents independently selected from OH, F, Br, Cl, CN, C₁₋₂alkyl, C₁₋₂fluoroalkyl, OC₁₋₂alkyl and OC₁₋₂fluoroalkyl. In an embodiment, R¹¹ is C₃₋₈cycloalkyl or C₃₋₈cycloalkenyl, each of which substituted with one or two substituents independently selected from OH, F, Br, Cl, CN, CH₃, CH₂CH₃, CF₃, OCH₃, and OCF₃.

In an embodiment, the compound of Formula (VI) is selected from

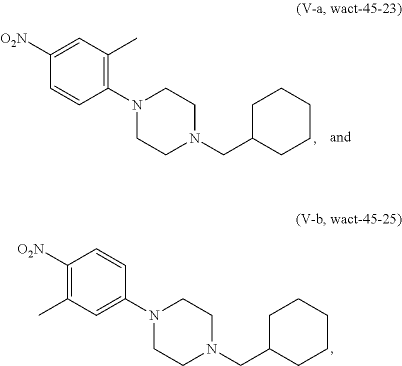 [SMILES: Cc1cc([N+](=O)[O-])ccc1N1CCN(CC2CCCCC2)CC1 ; Cc1cc(N2CCN(CC3CCCCC3)CC2)ccc1[N+](=O)[O-]]

and/or a pharmaceutically acceptable salt and/or solvate thereof.

In an embodiment, the present application includes methods for treating or preventing a nematode infection comprising administering an effective amount of a compound of Formula (VI)

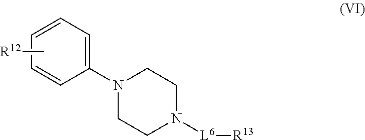

and/or a pharmaceutically acceptable salt and/or solvate thereof to a subject in need thereof
wherein:
R¹² is C₁₋₄alkyl, C₁₋₄fluoroalkyl, NO₂, Cl, F or CN;
L⁶ is a direct bond or C₁₋₄alkylene; and
R¹³ is C₃₋₈cycloalkyl or C₃₋₈cycloalkenyl, each of which is unsubstituted or substituted with one or two substituents independently selected from OH, F, Cl, Br, CN, C₁₋₄alkyl, C₁₋₄fluoroalkyl, OC₁₋₄alkyl, OC₁₋₄fluoroalkyl, and OC₁₋₄fluoroalkyl, provided when R¹² is NO₂ or CN and is in a position para to the piperazine on the phenyl ring, then R¹³ is not C₃₋₈cycloalkyl or C₃₋₈cycloalkenyl each of which is unsubstituted, or C₃₋₈cycloalkyl or C₃₋₈cycloalkenyl each of which is substituted with one or two substituents independently selected from C₁₋₄alkyl and C₁₋₄fluoroalkyl.

The application also includes a use of one or more compounds of Formula (VI) for treating or preventing a nematode infection as well as a use of one or more compounds of Formula (VI) for preparing a medicament for treating or preventing a nematode infection. The application further includes one or more compounds of Formula (VI) for use to treat or prevent a nematode infection.

In an embodiment, the present application includes methods of treating or preventing a disease, disorder or condition arising from a nematode infection comprising administering an effective amount a compound of Formula (VI)

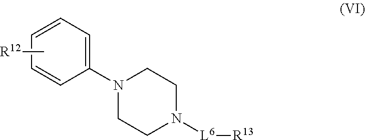

and/or a pharmaceutically acceptable salt and/or solvate thereof to a subject in need thereof
wherein:
R¹² is C₁₋₄alkyl, C₁₋₄fluoroalkyl, NO₂, Cl, F or CN;
L⁶ is a direct bond or C₁₋₄alkylene; and
R¹³ is C₃₋₈cycloalkyl or C₃₋₈cycloalkenyl, each of which is unsubstituted or substituted with one or two substituents independently selected from OH, F, Cl, Br, CN, C₁₋₄alkyl, C₁₋₄fluoroalkyl, OC₁₋₄alkyl and OC₁₋₄fluoroalkyl, provided when R¹² is NO₂ or CN and is in a position para to the piperazine on the phenyl ring, then R¹³ is not C₃₋₈cycloalkyl or C₃₋₈cycloalkenyl each of which is unsubstituted, or C₃₋₈cycloalkyl or C₃₋₈cycloalkenyl each of which is substituted with one or two substituents independently selected from C₁₋₄alkyl and C₁₋₄fluoroalkyl.

The application also includes a use of one or more compounds of Formula (VI) for treating or preventing a disease, disorder or condition arising from a nematode infection as well as a use of one or more compounds of Formula (VI) for preparing a medicament for treating or preventing a disease, disorder or condition arising from a nematode infection. The application further includes one or more compounds of Formula (VI) for use to treat or prevent a disease, disorder or condition arising from a nematode infection.

In an embodiment, R¹² is C₁₋₂alkyl, C₁₋₂fluoroalkyl, NO₂, Cl, F or CN. In an embodiment, R¹² is CH₃, CH₂CH₃, CF₃, NO₂, Cl, F or CN. In an embodiment, R¹² is CH₃, CF₃, NO₂, Cl, F, or CN. In an embodiment, R¹² is NO₂, CH₃ or CN and is in a position para to the piperazine on the phenyl ring. In an embodiment, R¹² is Cl or CF₃ and is in a position meta to the piperazine on the phenyl ring. In an embodiment, R¹² is F and is in a position ortho to the piperazine on the phenyl ring.

In an embodiment, L⁶ is a direct bond. In an embodiment, L⁶ is C₁₋₄alkylene. In an embodiment, L⁶ is C₁₋₃alkylene. In an embodiment, L⁶ is C₁alkylene or C₂alkylene. In an embodiment, L⁶ is C₁alkylene (CH₂).

In an embodiment, R¹³ is C₃₋₈cycloalkenyl. In an embodiment, the C₃₋₈ cycloalkenyl in R¹³ cyclopentenyl or cyclohexenyl. In an embodiment, the C₃₋₈ cycloalkenyl in R¹³ is cyclohexenyl. In an embodiment, the C₃₋₈cycloalkenyl in R¹³ is a bicyclic cycloalkenyl. In an embodiment, the bicyclic cycloalkenyl is bicycloheptenyl or bicyclooctenyl. In an embodiment, the bicyclic cycloalkenyl is bicycloheptenyl. In an embodiment, R¹³ is C₃₋₈cycloalkyl. In an embodiment, the C₃₋₈cycloalkyl in R¹³ is cyclobutyl, cyclopentyl, cyclohexyl, cycloheptyl or cyclooctyl. In an embodiment, the C₃₋₈ cycloalkyl in R¹³ is cyclopentyl, cyclohexyl, cycloheptyl or cyclooctyl. In an embodiment, the C₃₋₈cycloalkyl in R¹¹ is a bicyclic cycloalkyl. In an embodiment, the bicyclic cycloalkyl is bicycloheptyl or bicyclooctyl. In an embodiment, the bicyclic cycloalkyl is bicycloheptyl.

In an embodiment, R¹³ is C₃₋₈cycloalkyl or C₃₋₈cycloalkenyl each of which is unsubstituted. In an embodiment, R¹³ is C₃₋₈cycloalkyl or C₃₋₈cycloalkenyl each of which is substituted with one or two substituents independently selected from OH, F, Cl, Br, CN, C₁₋₄alkyl, C₁₋₄fluoroalkyl, OC₁₋₄alkyl and OC₁₋₄fluoroalkyl. In an embodiment, R¹³ is C₃₋₈ cycloalkyl or C₃₋₈cycloalkenyl, each of which substituted with one or two substituents independently selected from OH, F, Br, Cl, CN, C₁₋₂alkyl, C₁₋₂fluoroalkyl, OC₁₋₂alkyl and OC₁₋₂fluoroalkyl. In an embodiment, R¹³ is C₃₋₈cycloalkyl or C₃₋₈cycloalkenyl, each of which substituted with one or two substituents independently selected from OH, F, Br, Cl, CN, CH₃, CH₂CH₃, CF₃, OCH₃, and OCF₃. In an embodiment, R¹³ is C₃₋₈cycloalkyl or C₃₋₈ cycloalkenyl, each of which substituted with one substituent independently selected from OH, F, Br, Cl, CN, CH₃, CH₂CH₃, CF₃, OCH₃, and OCF₃. In an embodiment, R¹³ is C₃₋₈ cycloalkyl or C₃₋₈cycloalkenyl, each of which substituted with one substituent independently selected from F, CH₃ and CF₃. In an embodiment, R¹³ is C₃₋₈cycloalkyl which is substituted with one substituent independently selected from F, CH₃ and CF₃. In an embodiment, R¹³ is C₃₋₈cycloalkyl which is substituted with one substituent independently selected from F and CH₃.

In an embodiment, the compound of Formula (VI) is selected from

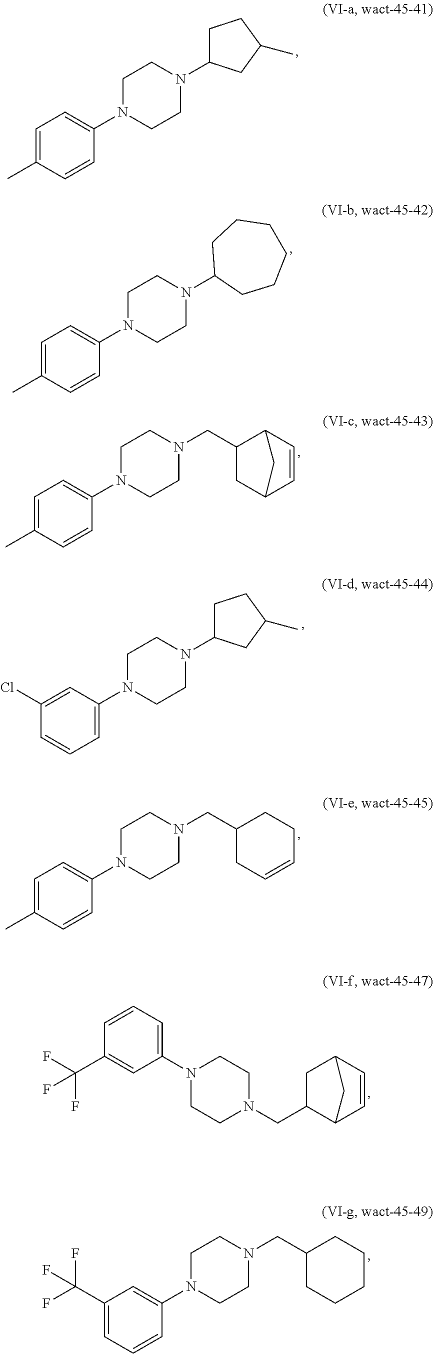

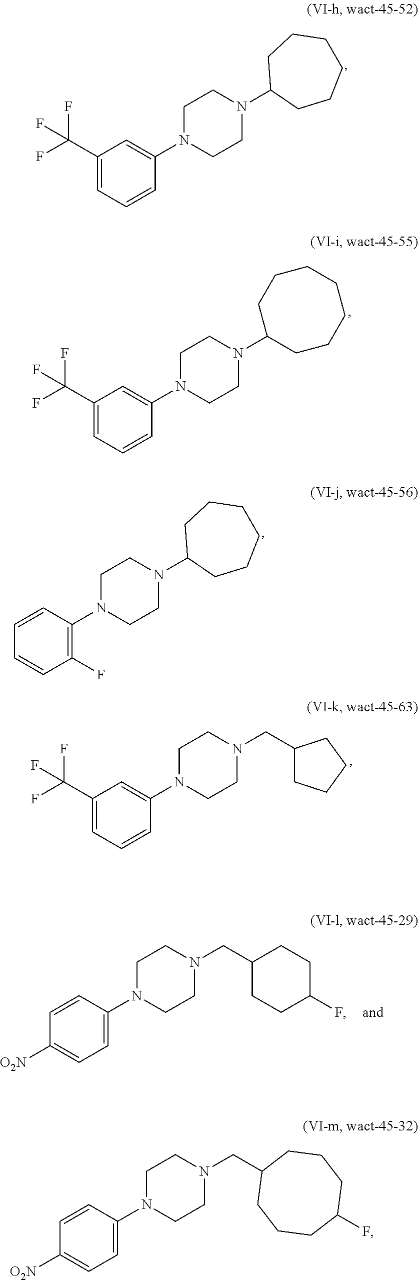 [SMILES: FC(F)(F)c1cccc(N2CCN(C3CCCCCC3)CC2)c1 ; FC(F)(F)c1cccc(N2CCN(C3CCCCCCC3)CC2)c1 ; Fc1ccccc1N1CCN(C2CCCCCC2)CC1 ; FC(F)(F)c1cccc(N2CCN(CC3CCCC3)CC2)c1 ; O=[N+]([O-])c1ccc(N2CCN(CC3CCC(F)CC3)CC2)cc1 ; O=[N+]([O-])c1ccc(N2CCN(CC3CCCC(F)CCC3)CC2)cc1]

and/or a pharmaceutically acceptable salt and/or solvate thereof.

In an embodiment, the compound of Formula (VI) is selected from

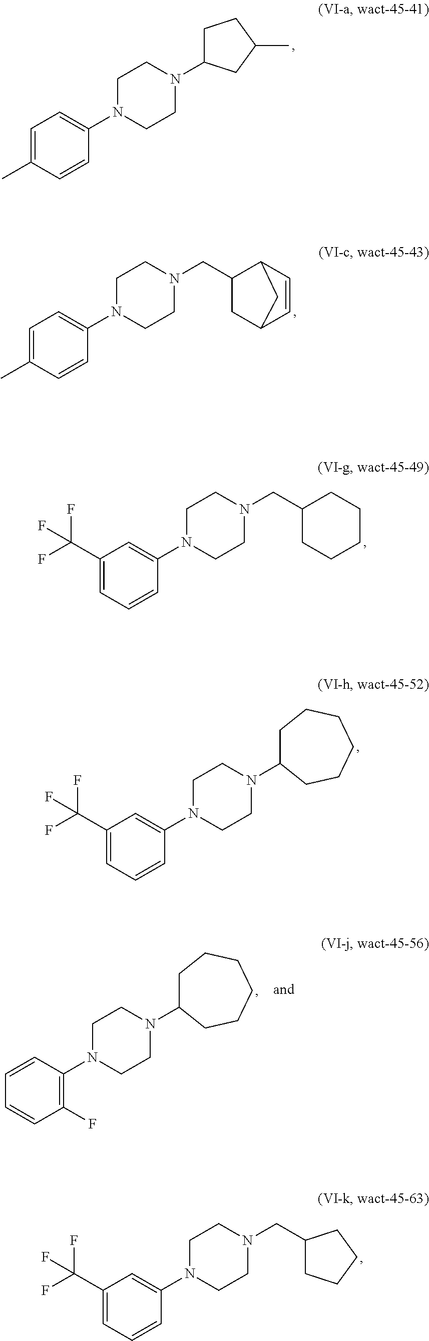

and/or a pharmaceutically acceptable salt and/or solvate thereof.

In an embodiment, the compound of Formula (VI) is selected from

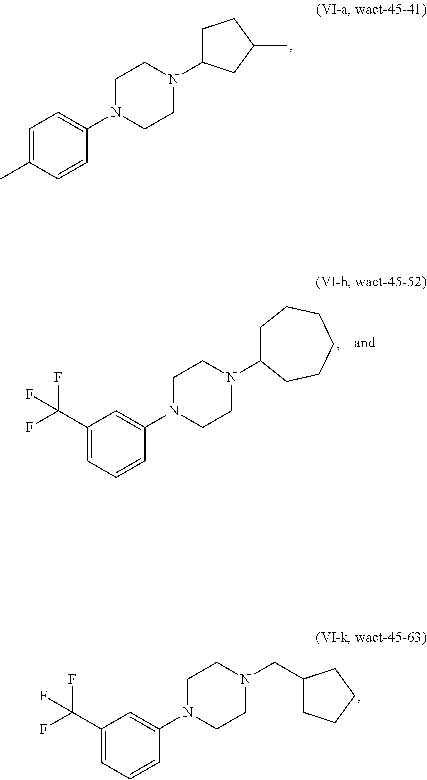

or a pharmaceutically acceptable salt and/or solvate thereof.

In an embodiment, the present application includes methods for treating or preventing a nematode infection or a disease, a disorder, or a condition arising from a nematode infection comprising administering an effective amount of a pharmaceutical composition comprising a compound of Formula (I), Formula (II), Formula (III), Formula (IV), Formula (V) and/or Formula (VI) described herein to a subject in need thereof.

In an embodiment, the present application includes uses of pharmaceutical compositions comprising a compound of Formula (I), Formula (II), Formula (III), Formula (IV), Formula (V) and/or Formula (VI) described herein for treating or preventing a nematode infection or a disease, a disorder, or a condition arising from a nematode infection in a subject in need thereof.

In an embodiment, the present application includes methods of treating or preventing a nematode infection comprising administering an effective amount of a nematicidal composition comprising a compound of Formula (I), Formula (II), Formula (III), Formula (IV), Formula (V) and/or Formula (VI) described herein to a subject in need thereof.

In an embodiment, the present application includes uses of a nematicidal composition comprising a compound of Formula (I), Formula (II), Formula (III), Formula (IV), Formula (V) and/or Formula (VI) described herein for treating or preventing a nematode infection or a disease, a disorder, or a condition arising from a nematode infection in a subject in need thereof.

In an embodiment, the nematode infection is an infection of a nematode of a species selected from Cooperia oncophora, Haemonchus contortus, and Caenorhabditis elegans.

Treatment methods comprise administering to a subject one or more compounds of the application, and optionally consists of a single administration, or alternatively comprises a series of administrations. The length of the treatment period depends on a variety of factors, such as the severity of the infection, disease, disorder or condition, the age of the subject, the dosage of the one or more compounds of the application, the activity of one or more compounds of the application, or a combination thereof.

In an embodiment, the one or more compounds of the application are administered or used as soon as possible after exposure to the nematode. In an embodiment, the one or more compounds of the application are administered or used until treatment of the nematode infection, disease disorder or condition is achieved. For example, until complete elimination of the nematode is achieved, or until the number of nematode has been reduced to the point where the subject's defenses are no longer overwhelmed and can kill any remaining nematode.

In an embodiment, the methods of the present application comprise administering an effective amount of a compound or a composition of the application to a subject selected from humans, mammals, birds, vertebrates, plants, seeds, and soil.

In an embodiment, the uses of the present application of a compound or a composition of the application are in a subject selected from humans, mammals, birds, vertebrates, plants, seeds, and soil.

In an embodiment, the nematode infects plants and the nematicidal composition is administered to the soil or to plants. In an embodiment, the nematicidal composition is administered to soil before planting. In an embodiment, the nematicidal composition is administered to soil after planting. In an embodiment, the nematicidal composition is administered to soil using a drip system. In an embodiment, the nematicidal composition is administered to soil using a drench system. In an embodiment, the nematicidal composition is administered to plant roots or plant foliage (e.g., leaves, stems). In some embodiments the nematicide composition is tilled into the soil or administered in furrow. In an embodiment, the nematicidal composition is administered to seeds.

In an embodiment, the nematode parasite infects a vertebrate. In an embodiment, the nematicidal composition is administered to non-human vertebrate. In an embodiment, the nematicidal composition is administered to a human. In an embodiment, the nematicidal composition is formulated as a drench to be administered to a non-human animal. In an embodiment, the nematicidal composition is formulated as an orally administered drug. In an embodiment, the nematicidal composition is formulated as an injectable drug. In an embodiment, the nematicidal composition is formulated for topical applications such as pour-ons, or for the use in tags or collars.

In an embodiment, the methods of the application comprise administering a compound or a composition of the application through one or more means selected from pre-planting, post-planting, as a feed additive, a drench, an external application, a pill and by injection.

In an embodiment, the present application includes methods of reducing the viability or fecundity or slowing the growth or development or inhibiting the infectivity of a nematode using a compound or a composition of the application as described herein.

In an embodiment, the present application includes methods of reducing the viability or fecundity or slowing the growth or development or inhibiting the infectivity of a nematode using a compound or a composition of the application as described herein, the methods comprising administering a compound or a composition of the application to subject selected from a human, a mammal, a bird, a vertebrate in general, a plant, a seed, or soil. In some examples, the bird can be a domesticated fowl, the mammal can be a domesticated animal and/or livestock.

The dosage of the one or more compounds of the application, varies depending on many factors such as the pharmacodynamic properties thereof, the mode of administration, the age, health and weight/mass of the subject, the nature and extent of the symptoms, the frequency of the treatment and the type of concurrent treatment, if any, and the clearance rate in the subject to be treated. One of skill in the art can determine the appropriate dosage based on the above factors. The one or more compounds of the application may be administered initially in a suitable dosage that may be adjusted as required, depending on the response.

Compounds can be tested for anthelmintic activity using methods known in the art. For example, the compound is combined with nematodes, e.g., in a well of microtiter dish, in liquid or solid media or in the soil containing the agent. Staged nematodes are placed on the media. The time of survival, viability of offspring, and/or the movement of the nematodes are measured. An agent with “anthelmintic or anthelminthic or antihelmthic activity” can, for example, reduce the survival time of adult nematodes relative to unexposed similarly staged adults, e.g., by about 20%, 40%, 60%, 80%, or more. In the alternative, an agent with “anthelmintic or anthelminthic or antihelminthic activity” may also cause the nematodes to cease replicating, regenerating, and/or producing viable progeny, e.g., by about 20%, 40%, 60%, 80%, or more. The effect may be apparent immediately or in successive generations.

### III. Compositions of the Application

In an embodiment, the present application includes pharmaceutical compositions comprising a pharmaceutically acceptable carrier and a compound of Formula (I)

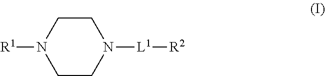

and/or a pharmaceutically acceptable salt and/or solvate thereof,
wherein:
R¹ is C₁₋₄alkyl;
R² is C₃₋₈cycloalkyl or C₃₋₈cycloalkenyl, each of which is unsubstituted or substituted with one or two substituents independently selected from OH, F, Cl, Br, CN, C₁₋₄alkyl, C₁₋₄fluoroalkyl, OC₁₋₄alkyl and OC₁₋₄fluoroalkyl; and
L¹ is a direct bond or C₁₋₄alkylene,
wherein the compound of Formula (I) is present in an amount effective to treat a nematode infection in a subject in need thereof.

In an embodiment, the present application includes a nematicidal composition comprising a carrier and a compound of Formula (I)

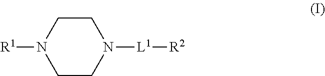

and/or a pharmaceutically acceptable salt and/or solvate thereof,
wherein
R¹ is C₁₋₄alkyl;
R² is C₃₋₈cycloalkyl or C₃₋₈cycloalkenyl, each of which is unsubstituted or substituted with one or two substituents independently selected from OH, F, Cl, Br, CN, C₁₋₄alkyl, C₁₋₄fluoroalkyl, OC₁₋₄alkyl and OC₁₋₄fluoroalkyl; and
L¹ is a direct bond or C₁₋₄alkylene, wherein the compound of Formula (I) is present in an amount effective to treat a nematode infection in a subject in need thereof.

In an embodiment, R¹ is C₁₋₃alkyl. In an embodiment, R¹ is methyl, ethyl, propyl or isopropyl. In an embodiment, R¹ is methyl or ethyl.

In an embodiment, R² is C₃₋₈cycloalkenyl. In an embodiment, the C₃₋₈ cycloalkenyl in R² is cyclopentenyl or cyclohexenyl. In an embodiment, the C₃₋₈ cycloalkenyl in R² is cyclohexenyl. In an embodiment, the C₃₋₈cycloalkenyl in R² is a bicyclic cycloalkenyl. In an embodiment, the bicyclic cycloalkenyl is bicycloheptenyl or bicyclooctenyl. In an embodiment, the bicyclic cycloalkenyl is bicycloheptenyl. In an embodiment, R² is C₃₋₈cycloalkyl. In an embodiment, the C₃₋₈cycloalkyl in R² is cyclobutyl, cyclopentyl or cyclohexyl. In an embodiment, the C₃₋₈cycloalkyl in R² is cyclohexyl. In an embodiment, the C₃₋₈cycloalkyl in R² is a bicyclic cycloalkyl. In an embodiment, the bicyclic cycloalkyl is bicycloheptyl or bicyclooctyl. In an embodiment, the bicyclic cycloalkyl is bicycloheptyl.

In an embodiment, R² is C₃₋₈cycloalkyl or C₃₋₈cycloalkenyl each of which is unsubstituted. In an embodiment, R² is C₃₋₈cycloalkyl or C₃₋₈cycloalkenyl each of which is substituted with one or two substituents independently selected from OH, F, Cl, Br, CN, C₁₋₄alkyl, C₁₋₄fluoroalkyl, OC₁₋₄alkyl and OC₁₋₄fluoroalkyl. In an embodiment, R² is C₃₋₈ cycloalkyl or C₃₋₈cycloalkenyl, each of which substituted with one or two substituents independently selected from OH, F, Br, Cl, CN, C₁₋₂alkyl, C₁₋₂fluoroalkyl, OC₁₋₂alkyl and OC₁₋₂fluoroalkyl. In an embodiment, R² is C₃₋₈cycloalkyl or C₃₋₈cycloalkenyl, each of which substituted with one or two substituents independently selected from OH, F, Br, Cl, CN, CH₃, CH₂CH₃, CF₃, OCH₃, and OCF₃.

In an embodiment, L¹ is a direct bond. In an embodiment, L¹ is C₁₋₄alkylene. In an embodiment, L¹ is C₁₋₃alkylene. In an embodiment, L¹ is C₁alkylene or C₂alkylene. In an embodiment, L¹ is C₁alkylene.

In an embodiment, the compound of Formula (I) is

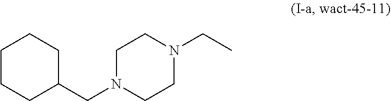

or a pharmaceutically acceptable salt and/or solvate thereof.

In an embodiment, the present application includes pharmaceutical compositions comprising a pharmaceutically acceptable carrier and a compound of Formula (III)

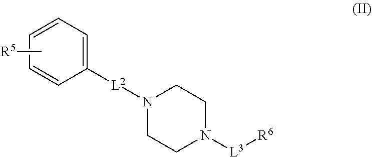

and/or a pharmaceutically acceptable salt and/or solvate thereof,
wherein:
R⁵ is C₁₋₄alkyl, C₁₋₄fluoroalkyl, NO₂, Cl, F or CN;
L² is C₁₋₄alkylene;
L³ is a direct bond or C₁₋₄alkylene; and
R⁸ is C₃₋₈cycloalkyl or C₃₋₈cycloalkenyl, each of which is unsubstituted or substituted with one or two substituents independently selected from OH, F, Cl, Br, CN, C₁₋₄alkyl, C₁₋₄fluoroalkyl, OC₁₋₄alkyl and OC₁₋₄fluoroalkyl,
wherein the compound of Formula (II) is present in an amount effective to treat a nematode infection in a subject in need thereof.

In an embodiment, the present application includes a nematicidal composition comprising a carrier and a compound of Formula (II)

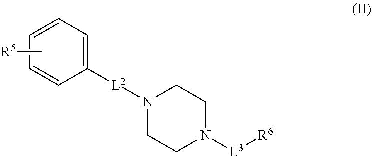

and/or a pharmaceutically acceptable salt and/or solvate thereof
wherein:
R⁵ is C₁₋₄alkyl, C₁₋₄fluoroalkyl, NO₂, Cl, F or CN;
L² is C₁₋₄alkylene;
L³ is a direct bond or C₁₋₄alkylene; and
R⁶ is C₃₋₈cycloalkyl or C₃₋₈cycloalkenyl, each of which is unsubstituted or substituted with one or two substituents independently selected from OH, F, Cl, Br, CN, C₁₋₄alkyl, C₁₋₄fluoroalkyl, OC₁₋₄alkyl and OC₁₋₄fluoroalkyl,
wherein the compound of Formula (II) is present in an amount effective to treat a nematode infection in a subject in need thereof.

In an embodiment, R⁵ is C₁₋₂alkyl, C₁₋₂fluoroalkyl, NO₂, Cl, F or CN. In an embodiment, R⁵ is CH₃, CH₂CH₃, CF₃, NO₂, Cl, F or CN. In an embodiment, R⁵ is CH₃, CH₂CH₃, or CF₃. In an embodiment, R⁵ is NO₂, F or CN. In an embodiment, R⁵ is NO₂. In an embodiment, R⁵ is NO₂ and is in a position para to the piperazine on the phenyl ring. In an embodiment, R⁵ is NO₂ and is in a position meta to the piperazine on the phenyl ring.

In an embodiment, L² is C₁₋₃alkylene. In an embodiment, L² is C₁alkylene or C₂alkylene. In an embodiment, L² is C₁alkylene (CH₂).

In an embodiment, L³ is a direct bond. In an embodiment, L³ is C₁₋₄alkylene. In an embodiment, L³ is C₁₋₃alkylene. In an embodiment, L³ is C₁alkylene or C₂alkylene. In an embodiment, L³ is C₁alkylene (CH₂).

In an embodiment, R⁶ is C₃₋₈cycloalkenyl. In an embodiment, the C₃₋₈ cycloalkenyl in R⁶ is cyclopentenyl or cyclohexenyl. In an embodiment, the C₃₋₈ cycloalkenyl in R⁶ is a bicyclic cycloalkenyl. In an embodiment, the bicyclic cycloalkenyl is bicycloheptenyl or bicyclooctenyl. In an embodiment, the bicyclic cycloalkenyl is bicycloheptenyl. In an embodiment, R⁶ is C₃₋₈cycloalkyl. In an embodiment, the C₃₋₈ cycloalkyl in R⁶ is cyclobutyl, cyclopentyl or cyclohexyl. In an embodiment, the C₃₋₈ cycloalkyl in R⁶ is cyclopentyl or cyclohexyl. In an embodiment, the C₃₋₈cycloalkyl in R⁶ is a bicyclic cycloalkyl. In an embodiment, the bicyclic cycloalkyl is bicycloheptyl or bicyclooctyl. In an embodiment, the bicyclic cycloalkyl is bicycloheptyl.

In an embodiment, R⁶ is C₃₋₈cycloalkyl or C₃₋₈cycloalkenyl each of which is unsubstituted. In an embodiment, R⁶ is C₃₋₈cycloalkyl or C₃₋₈cycloalkenyl each of which is substituted with one or two substituents independently selected from OH, F, Cl, Br, CN, C₁₋₄alkyl, C₁₋₄fluoroalkyl, OC₁₋₄alkyl and OC₁₋₄fluoroalkyl. In an embodiment, R⁶ is C₃₋₈ cycloalkyl or C₃₋₈cycloalkenyl, each of which substituted with one or two substituents independently selected from OH, F, Br, Cl, CN, C₁₋₂alkyl, C₁₋₂fluoroalkyl, OC₁₋₂alkyl and OC₁₋₂fluoroalkyl. In an embodiment, R⁶ is C₃₋₈cycloalkyl or C₃₋₈cycloalkenyl, each of which substituted with one or two substituents independently selected from OH, F, Br, Cl, CN, CH₃, CH₂CH₃, CF₃, OCH₃, and OCF₃.

In an embodiment, the compound of Formula (III) is selected from

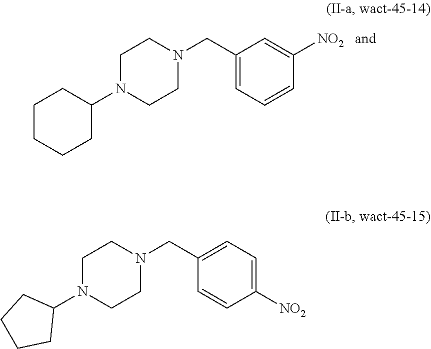 [SMILES: O=[N+]([O-])c1cccc(CN2CCN(C3CCCCC3)CC2)c1 ; "and" ; O=[N+]([O-])c1ccc(CN2CCN(C3CCCC3)CC2)cc1]

or a pharmaceutically acceptable salt and/or solvate thereof.

In an embodiment, the present application includes pharmaceutical compositions comprising a pharmaceutically acceptable carrier and a compound of Formula (IV)

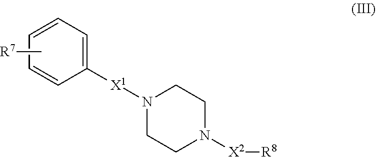

and/or a pharmaceutically acceptable salt and/or solvate thereof,
wherein:
R⁷ is H, C₁₋₄alkyl, C₁₋₄fluoroalkyl, NO₂, Cl, F or CN;
one of X¹ or X² is C═O and the other is a direct bond; and
R⁸ is phenyl, C₃₋₈cycloalkyl or C₃₋₈cycloalkenyl, each of which is unsubstituted or substituted with one or two substituents independently selected from OH, F, Cl, Br, CN, C₁₋₄alkyl, C₁₋₄fluoroalkyl, OC₁₋₄alkyl and OC₁₋₄fluoroalkyl,
wherein the compound of Formula (III) is present in an amount effective to treat a nematode infection in a subject in need thereof.

In an embodiment, the present application includes a nematicidal composition comprising a carrier and a compound of Formula (III)

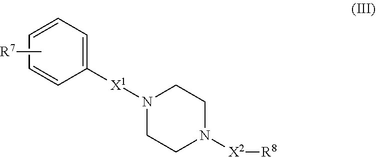

and/or a pharmaceutically acceptable salt and/or solvate thereof,
wherein:
R⁷ is H, C₁₋₄alkyl, C₁₋₄fluoroalkyl, NO₂, Cl, F or CN;
one of X¹ or X² is C═O and the other is a direct bond; and
R⁸ is phenyl, C₃₋₈cycloalkyl or C₃₋₈cycloalkenyl, each of which is unsubstituted or substituted with one or two substituents independently selected from OH, F, Cl, Br, CN, C₁₋₄alkyl, C₁₋₄fluoroalkyl, OC₁₋₄alkyl and OC₁₋₄fluoroalkyl,
wherein the compound of Formula (III) is present in an amount effective to treat a nematode infection in a subject in need thereof.

In an embodiment, R⁷ is H, C₁₋₂alkyl, C₁₋₂fluoroalkyl, NO₂, Cl, F or CN. In an embodiment, R⁷ is H, CH₃, CH₂CH₃, CF₃, NO₂, Cl, F or CN. In an embodiment, R⁷ is H, CH₃, CH₂CH₃, or CF₃. In an embodiment, R⁷ is H. In an embodiment, R⁷ is CH₃, CH₂CH₃, or CF₃. In an embodiment, R⁷ is NO₂, F or CN. In an embodiment, R⁷ is NO₂ or F.

In an embodiment, X¹ is C═O and X² is a direct bond. In an embodiment, X¹ is a direct bond and X² is C═O.

In an embodiment, R⁸ is phenyl. In an embodiment, R⁸ is C₃₋₈cycloalkenyl. In an embodiment, the C₃₋₈cycloalkenyl in R⁸ is cyclopentenyl or cyclohexenyl. In an embodiment, the C₃₋₈cycloalkenyl in R⁸ is a bicyclic cycloalkenyl. In an embodiment, the bicyclic cycloalkenyl is bicycloheptenyl or bicyclooctenyl. In an embodiment, the bicyclic cycloalkenyl is bicycloheptenyl. In an embodiment, R⁸ is C₃₋₈cycloalkyl. In an embodiment, the C₃₋₈cycloalkyl in R⁸ is cyclobutyl, cyclopentyl or cyclohexyl. In an embodiment, the C₃₋₈ cycloalkyl in R⁸ is a bicyclic cycloalkyl. In an embodiment, the bicyclic cycloalkyl is bicycloheptyl or bicyclooctyl. In an embodiment, the bicyclic cycloalkyl is bicycloheptyl.

In an embodiment, R⁸ is phenyl, C₃₋₈cycloalkyl or C₃₋₈cycloalkenyl, each of which is unsubstituted. In an embodiment, R⁸ is phenyl, C₃₋₈cycloalkyl or C₃₋₈cycloalkenyl, each of which is substituted with one or two substituents independently selected from OH, F, Cl, Br, CN, C₁₋₄alkyl, C₁₋₄fluoroalkyl, OC₁₋₄alkyl and OC₁₋₄fluoroalkyl. In an embodiment, R⁸ is phenyl, C₃₋₈cycloalkyl or C₃₋₈cycloalkenyl, each of which substituted with one or two substituents from OH, F, Br, Cl, CN, C₁₋₂alkyl, C₁₋₂fluoroalkyl, OC₁₋₂alkyl and OC₁₋₂fluoroalkyl. In an embodiment, R⁸ is phenyl, C₃₋₈cycloalkyl or C₃₋₈cycloalkenyl, each of which substituted with one or two substituents selected from OH, F, Br, Cl, CN, CH₃, CH₂CH₃, CF₃, OCH₃, and OCF₃. In an embodiment, R⁸ is phenyl which substituted with one or two substituents independently selected from OH, F, Br, Cl, and CF₃. In an embodiment, R⁸ is phenyl which substituted Br.

In an embodiment, the compound of Formula (III) is selected from

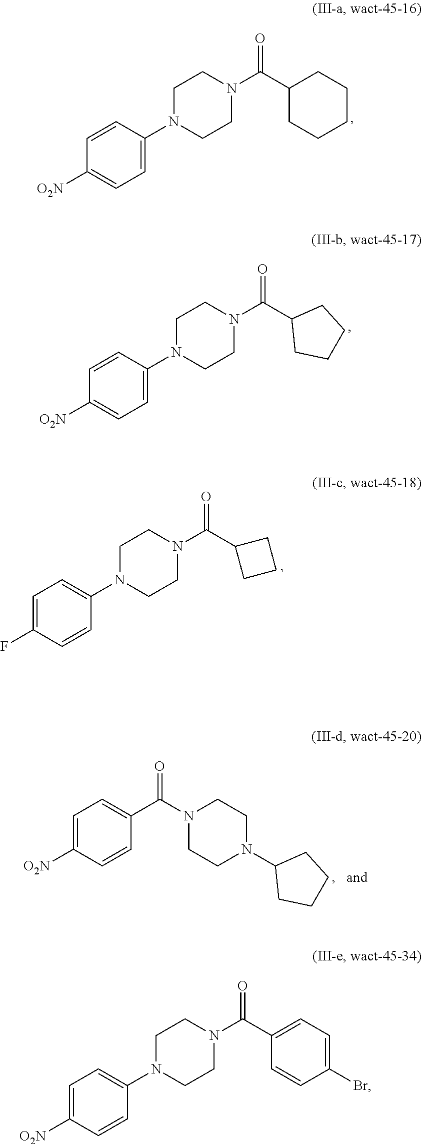

or a pharmaceutically acceptable salt and/or solvate thereof.

In an embodiment, the present application includes pharmaceutical compositions comprising a pharmaceutically acceptable carrier and a compound of Formula (IV)

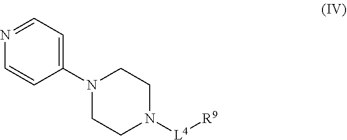

and/or a pharmaceutically acceptable salt and/or solvate thereof,
wherein L⁴ is a direct bond or C₁₋₄alkylene; and
R⁹ is C₃₋₈cycloalkyl or C₃₋₈cycloalkenyl, each of which is unsubstituted or substituted with
one or two substituents independently selected from OH, F, Cl, Br, CN, C₁₋₄alkyl, C₁₋₄fluoroalkyl, OC₁₋₄alkyl and OC₁₋₄fluoroalkyl,
wherein the compound of Formula (IV) is present in an amount effective to treat a nematode infection in a subject in need thereof.

In an embodiment, the present application includes a nematicidal composition comprising a carrier and a compound of Formula (IV)

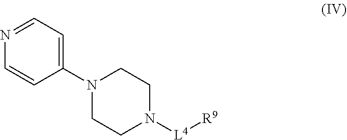

and/or a pharmaceutically acceptable salt and/or solvate thereof,
wherein
L⁴ is a direct bond or C₁₋₄alkylene; and
R⁹ is C₃₋₈cycloalkyl or C₃₋₈cycloalkenyl, each of which is unsubstituted or substituted with one or two substituents independently selected from OH, F, Cl, Br, CN, C₁₋₄alkyl, C₁₋₄fluoroalkyl, OC₁₋₄alkyl and OC₁₋₄fluoroalkyl,
wherein the compound of Formula (IV) is present in an amount effective to treat a nematode infection in a subject in need thereof.

In an embodiment, L⁴ is a direct bond. In an embodiment, L⁴ is C₁₋₄alkylene. In an embodiment, L⁴ is C₁₋₃alkylene. In an embodiment, L⁴ is C₁alkylene or C₂alkylene. In an embodiment, L⁴ is C₁alkylene (CH₂).

In an embodiment, R⁹ is C₃₋₈cycloalkenyl. In an embodiment, the C₃₋₈ cycloalkenyl in R⁹ is cyclopentenyl or cyclohexenyl. In an embodiment, the C₃₋₈ cycloalkenyl in R⁹ is a bicyclic cycloalkenyl. In an embodiment, the bicyclic cycloalkenyl is bicycloheptenyl or bicyclooctenyl. In an embodiment, the bicyclic cycloalkenyl is bicycloheptenyl. In an embodiment, R⁹ is C₃₋₈cycloalkyl. In an embodiment, the C₃₋₈ cycloalkyl in R⁹ is cyclobutyl, cyclopentyl or cyclohexyl. In an embodiment, R⁹ is cyclopentyl or cyclohexyl. In an embodiment, the C₃₋₈cycloalkyl in R⁹ is a bicyclic cycloalkyl. In an embodiment, the bicyclic cycloalkyl is bicycloheptyl or bicyclooctyl. In an embodiment, the bicyclic cycloalkyl is bicycloheptyl.

In an embodiment, R⁹ is C₃₋₈cycloalkyl or C₃₋₈cycloalkenyl each of which is unsubstituted. In an embodiment, R⁹ is C₃₋₈cycloalkyl or C₃₋₈cycloalkenyl each of which is substituted with one or two substituents independently selected from OH, F, Cl, Br, CN, C₁₋₄alkyl, C₁₋₄fluoroalkyl, OC₁₋₄alkyl and OC₁₋₄fluoroalkyl. In an embodiment, R⁹ is C₃₋₈ cycloalkyl or C₃₋₈cycloalkenyl, each of which substituted with one or two substituents independently selected from OH, F, Br, Cl, CN, C₁₋₂alkyl, C₁₋₂fluoroalkyl, OC₁₋₂alkyl and OC₁₋₂fluoroalkyl. In an embodiment, R⁹ is C₃₋₈cycloalkyl or C₃₋₈cycloalkenyl, each of which substituted with one or two substituents independently selected from OH, F, Br, Cl, CN, CH₃, CH₂CH₃, CF₃, OCH₃, and OCF₃.

In an embodiment, the compound of Formula (IV) is selected from

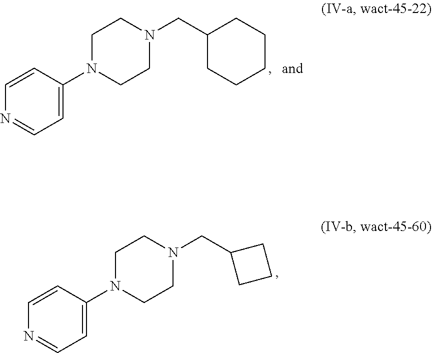 [SMILES: c1cc(N2CCN(CC3CCCCC3)CC2)ccn1 ; "," ; "and" ; c1cc(N2CCN(CC3CCC3)CC2)ccn1 ; ","]

or a pharmaceutically acceptable salt and/or solvate thereof.

In an embodiment, the present application includes pharmaceutical compositions comprising a pharmaceutically acceptable carrier and a compound of Formula (V)

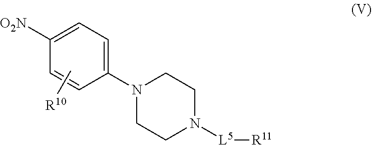

and/or a pharmaceutically acceptable salt and/or solvate thereof,
wherein:
R¹⁰ is C₁₋₄alkyl, C₁₋₄fluoroalkyl, Cl, F or CN;
L⁵ is a direct bond or C₁₋₄alkylene; and
R¹¹ is C₃₋₈cycloalkyl or C₃₋₈cycloalkenyl, each of which is unsubstituted or substituted with one or two substituents independently selected from OH, F, Cl, Br, CN, C₁₋₄alkyl, C₁₋₄fluoroalkyl, OC₁₋₄alkyl and OC₁₋₄fluoroalkyl,
wherein the compound of Formula (V) is present in an amount effective to treat a nematode infection in a subject in need thereof.

In an embodiment, the present application includes a nematicidal composition comprising a carrier and a compound of Formula (V)

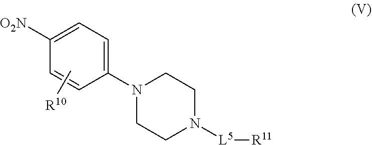

and/or a pharmaceutically acceptable salt and/or solvate thereof,
wherein:
R¹⁰ is C₁₋₄alkyl, C₁₋₄fluoroalkyl, Cl, F or CN;
L⁵ is a direct bond or C₁₋₄alkylene; and
R¹¹ is C₃₋₈cycloalkyl or C₃₋₈cycloalkenyl, each of which is unsubstituted or substituted with one or two substituents independently selected from OH, F, Cl, Br, CN, C₁₋₄alkyl, C₁₋₄fluoroalkyl, OC₁₋₄alkyl and OC₁₋₄fluoroalkyl,
wherein the compound of Formula (V) is present in an amount effective to treat a nematode infection in a subject in need thereof.

In an embodiment, R¹⁰ is C₁₋₂alkyl, C₁₋₂fluoroalkyl, Cl, F or CN. In an embodiment, R¹⁰ is CH₃, CH₂CH₃, CF₃, CI, F or CN. In an embodiment, R¹⁰ is CH₃, CH₂CH₃, Cl, F, CN or CF₃. In an embodiment, R¹⁰ is CH₃, CH₂CH₃ or CF₃. In an embodiment, R¹⁰ is CH₃ and is in a position meta to the piperazine on the phenyl ring, and is in a position ortho to the piperazine on the phenyl ring.

In an embodiment, L⁵ is a direct bond. In an embodiment, L⁵ is C₁₋₄alkylene. In an embodiment, L⁵ is C₁₋₃alkylene. In an embodiment, L⁵ is C₁alkylene or C₂alkylene. In an embodiment, L⁵ is C₁alkylene (CH₂).

In an embodiment, R¹¹ is C₃₋₈cycloalkenyl. In an embodiment, the C₃₋₈ cycloalkenyl in R¹¹ cyclopentenyl or cyclohexenyl. In an embodiment, the C₃₋₈ cycloalkenyl in R¹¹ is a bicyclic cycloalkenyl. In an embodiment, the bicyclic cycloalkenyl is bicycloheptenyl or bicyclooctenyl. In an embodiment, the bicyclic cycloalkenyl is bicycloheptenyl. In an embodiment, R¹¹ is C₃₋₈cycloalkyl. In an embodiment, the C₃₋₈ cycloalkyl in R¹¹ is cyclobutyl, cyclopentyl or cyclohexyl. In an embodiment, the C₃₋₈ cycloalkyl in R¹¹ is cyclohexyl. In an embodiment, the C₃₋₈cycloalkyl in R¹¹ is a bicyclic cycloalkyl. In an embodiment, the bicyclic cycloalkyl is bicycloheptyl or bicyclooctyl. In an embodiment, the bicyclic cycloalkyl is bicycloheptyl.

In an embodiment, R¹¹ is C₃₋₈cycloalkyl or C₃₋₈cycloalkenyl each of which is unsubstituted. In an embodiment, R¹¹ is C₃₋₈cycloalkyl or C₃₋₈cycloalkenyl each of which is substituted with one or two substituents independently selected from OH, F, Cl, Br, CN, C₁₋₄alkyl, C₁₋₄fluoroalkyl, OC₁₋₄alkyl and OC₁₋₄fluoroalkyl. In an embodiment, R¹¹ is C₃₋₈ cycloalkyl or C₃₋₈cycloalkenyl, each of which substituted with one or two substituents independently selected from OH, F, Br, Cl, CN, C₁₋₂alkyl, C₁₋₂fluoroalkyl, OC₁₋₂alkyl and OC₁₋₂fluoroalkyl. In an embodiment, R¹¹ is C₃₋₈cycloalkyl or C₃₋₈cycloalkenyl, each of which substituted with one or two substituents independently selected from OH, F, Br, Cl, CN, CH₃, CH₂CH₃, CF₃, OCH₃, and OCF₃.

In an embodiment, the compound of Formula (V) is selected from

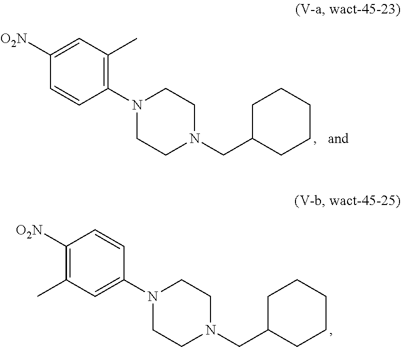 [SMILES: Cc1cc([N+](=O)[O-])ccc1N1CCN(CC2CCCCC2)CC1 ; "," ; "and" ; Cc1cc(N2CCN(CC3CCCCC3)CC2)ccc1[N+](=O)[O-] ; ","]

or a pharmaceutically acceptable salt and/or solvate thereof.

In an embodiment, the present application includes pharmaceutical compositions comprising a pharmaceutically acceptable carrier and a compound of Formula (VI)

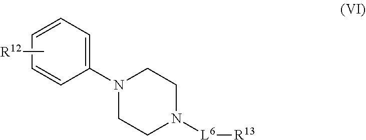

and/or a pharmaceutically acceptable salt and/or solvate thereof,
wherein:
R¹² is C₁₋₄alkyl, C₁₋₄fluoroalkyl, NO₂, Cl, F or CN;
L⁶ is a direct bond or C₁₋₄alkylene; and
R¹³ is C₃₋₈cycloalkyl or C₃₋₈cycloalkenyl, each of which is unsubstituted or substituted with one or two substituents independently selected from OH, F, Cl, Br, CN, C₁₋₄alkyl, C₁₋₄fluoroalkyl, OC₁₋₄alkyl, OC₁₋₄fluoroalkyl, and OC₁₋₄fluoroalkyl,
provided when R¹² is NO₂ or CN and is in a position para to the piperazine on the phenyl ring, then R¹³ is not C₃₋₈cycloalkyl or C₃₋₈cycloalkenyl each of which is unsubstituted, or C₃₋₈cycloalkyl or C₃₋₈cycloalkenyl each of which is substituted with one or two substituents independently selected from C₁₋₄alkyl and C₁₋₄fluoroalkyl,
wherein the compound of Formula (VI) is present in an amount effective to treat a nematode infection in a subject in need thereof.

In an embodiment, the present application includes a nematicidal composition comprising a carrier and a compound of Formula (VI)

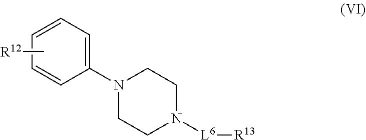

and/or a pharmaceutically acceptable salt and/or solvate thereof,
wherein:
R¹² is C₁₋₄alkyl, C₁₋₄fluoroalkyl, NO₂, Cl, F or CN;
L⁶ is a direct bond or C₁₋₄alkylene; and
R¹³ is C₃₋₈cycloalkyl or C₃₋₈cycloalkenyl, each of which is unsubstituted or substituted with one or two substituents independently selected from OH, F, Cl, Br, CN, C₁₋₄alkyl, C₁₋₄fluoroalkyl, OC₁₋₄alkyl, OC₁₋₄fluoroalkyl, and OC₁₋₄fluoroalkyl,
provided when R¹² is NO₂ or CN and is in a position para to the piperazine on the phenyl ring, then R¹³ is not C₃₋₈cycloalkyl or C₃₋₈cycloalkenyl each of which is unsubstituted, or C₃₋₈cycloalkyl or C₃₋₈cycloalkenyl each of which is substituted with one or two substituents independently selected from C₁₋₄alkyl and C₁₋₄fluoroalkyl,
wherein the compound of Formula (VI) is present in an amount effective to treat a nematode infection in a subject in need thereof.

In an embodiment, R¹² is C₁₋₂alkyl, C₁₋₂fluoroalkyl, NO₂, Cl, F or CN. In an embodiment, R¹² is CH₃, CH₂CH₃, CF₃, NO₂, Cl, F or CN. In an embodiment, R¹² is CH₃, CF₃, NO₂, CI, F, or CN. In an embodiment, R¹² is NO₂, CH₃ or CN and is in a position para to the piperazine on the phenyl ring. In an embodiment, R¹² is C or CF₃ and is in a position meta to the piperazine on the phenyl ring. In an embodiment, R¹² is F and is in a position ortho to the piperazine on the phenyl ring.

In an embodiment, L⁶ is a direct bond. In an embodiment, L⁶ is C₁₋₄alkylene. In an embodiment, L⁶ is C₁₋₃alkylene. In an embodiment, L⁶ is C₁alkylene or C₂alkylene. In an embodiment, L⁶ is C₁alkylene (CH₂).

In an embodiment, R¹³ is C₃₋₈cycloalkenyl. In an embodiment, the C₃₋₈cycloalkenyl in R¹³ cyclopentenyl or cyclohexenyl. In an embodiment, the C₃₋₈cycloalkenyl in R¹³ is cyclohexenyl. In an embodiment, the C₃₋₈cycloalkenyl in R¹³ is a bicyclic cycloalkenyl. In an embodiment, the bicyclic cycloalkenyl is bicycloheptenyl or bicyclooctenyl. In an embodiment, the bicyclic cycloalkenyl is bicycloheptenyl. In an embodiment, R¹³ is C₃₋₈cycloalkyl. In an embodiment, the C₃₋₈cycloalkyl in R¹³ is cyclobutyl, cyclopentyl, cyclohexyl, cycloheptyl or cyclooctyl. In an embodiment, the C₃₋₈cycloalkyl in R¹³ is cyclopentyl, cyclohexyl, cycloheptyl or cyclooctyl. In an embodiment, the C₃₋₈cycloalkyl in R¹¹ is a bicyclic cycloalkyl. In an embodiment, the bicyclic cycloalkyl is bicycloheptyl or bicyclooctyl. In an embodiment, the bicyclic cycloalkyl is bicycloheptyl.

In an embodiment, R¹³ is C₃₋₈cycloalkyl or C₃₋₈cycloalkenyl each of which is unsubstituted. In an embodiment, R¹³ is C₃₋₈cycloalkyl or C₃₋₈cycloalkenyl each of which is substituted with one or two substituents independently selected from OH, F, Cl, Br, CN, C₁₋₄alkyl, C₁₋₄fluoroalkyl, OC₁₋₄alkyl and OC₁₋₄fluoroalkyl. In an embodiment, R¹³ is C₃₋₈cycloalkyl or C₃₋₈cycloalkenyl, each of which substituted with one or two substituents independently selected from OH, F, Br, Cl, CN, C₁₋₂alkyl, C₁₋₂fluoroalkyl, OC₁₋₂alkyl and OC₁₋₂fluoroalkyl. In an embodiment, R¹³ is C₃₋₈cycloalkyl or C₃₋₈cycloalkenyl, each of which substituted with one or two substituents independently selected from OH, F, Br, Cl, CN, CH₃, CH₂CH₃, CF₃, OCH₃, and OCF₃. In an embodiment, R¹³ is C₃₋₈cycloalkyl or C₃₋₈cycloalkenyl, each of which substituted with one substituent independently selected from OH, F, Br, Cl, CN, CH₃, CH₂CH₃, CF₃, OCH₃, and OCF₃. In an embodiment, R¹³ is C₃₋₈cycloalkyl or C₃₋₈cycloalkenyl, each of which substituted with one substituent independently selected from F, CH₃ and CF₃. In an embodiment, R¹³ is C₃₋₈cycloalkyl which is substituted with one substituent independently selected from F, CH₃ and CF₃. In an embodiment, R¹³ is C₃₋₈cycloalkyl which is substituted with one substituent independently selected from F and CH₃.

In an embodiment, the compound of Formula (VI) is selected from

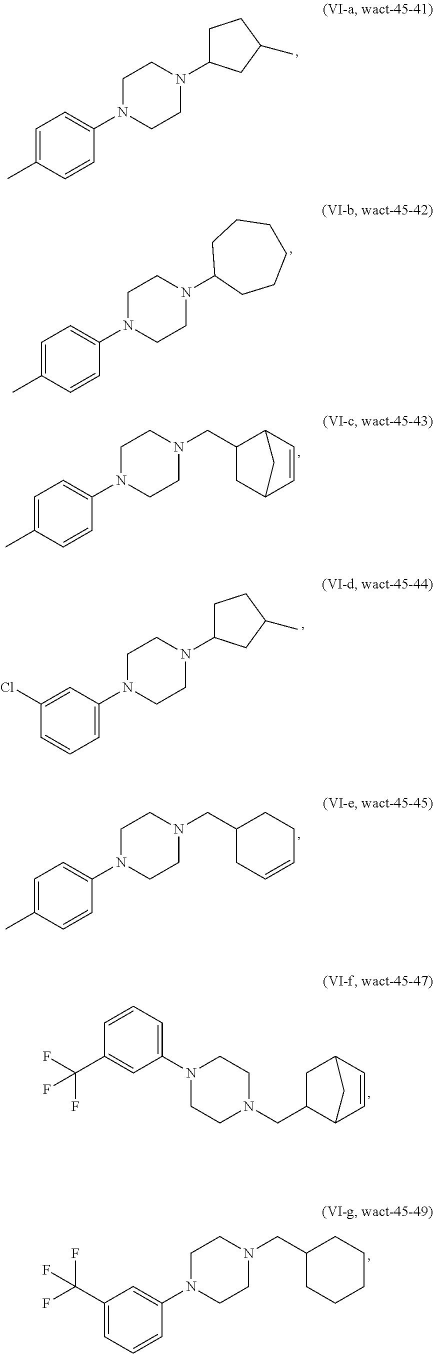

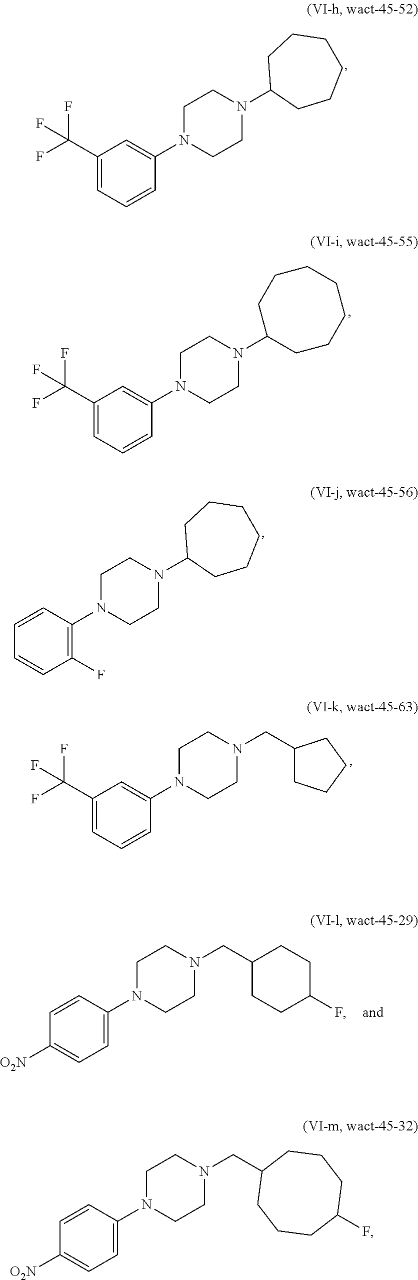 [SMILES: FC(F)(F)c1cccc(N2CCN(C3CCCCCC3)CC2)c1 ; FC(F)(F)c1cccc(N2CCN(C3CCCCCCC3)CC2)c1 ; Fc1ccccc1N1CCN(C2CCCCCC2)CC1 ; FC(F)(F)c1cccc(N2CCN(CC3CCCC3)CC2)c1 ; O=[N+]([O-])c1ccc(N2CCN(CC3CCC(F)CC3)CC2)cc1 ; O=[N+]([O-])c1ccc(N2CCN(CC3CCCC(F)CCC3)CC2)cc1]

or a pharmaceutically acceptable salt and/or solvate thereof.

In an embodiment, the compound of Formula (VI) is selected from

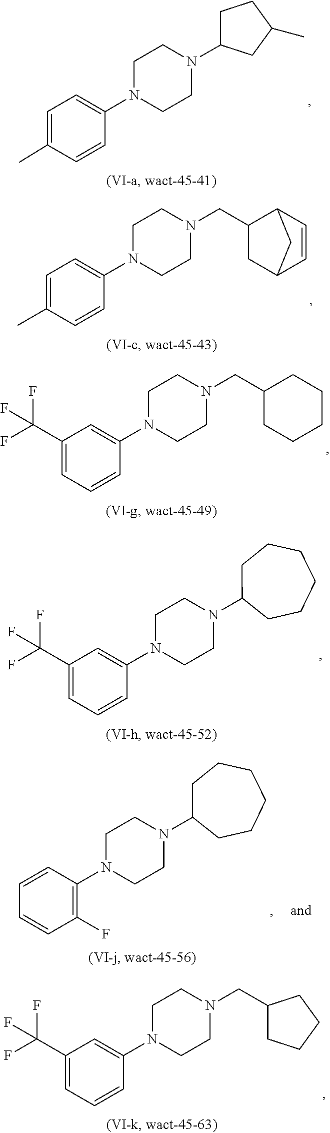

and/or a pharmaceutically acceptable salt and/or solvate thereof.

In an embodiment, the compound of Formula (VI) is selected from

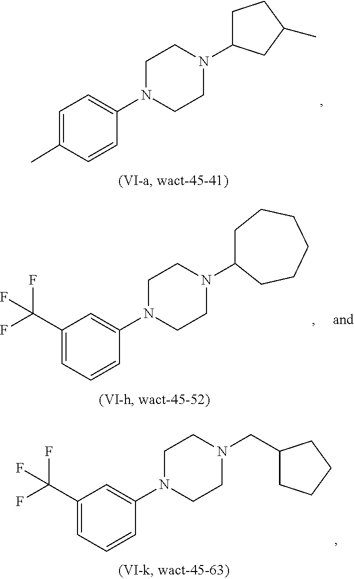

or a pharmaceutically acceptable salt and/or solvate thereof.

In an embodiment, the present application includes uses of pharmaceutical compositions comprising a compound of Formula (I), Formula (II), Formula (III), Formula (IV), Formula (V) and/or Formula (VI) for treating or preventing a nematode infection or a disease, a disorder, or a condition arising from a nematode infection in a subject in need thereof.

In an embodiment, the nematode infection is an infection of a nematode of the following non-limiting, exemplary genera: Caenorhabditis, Nippostrongyles, Anguina, Ditylenchus, Tylenchorhynchus, Pratylenchus, Radopholus, Hirschmanniella, Nacobbus, Hoplolaimus, Scutellonema, Rotylenchus, Helicotylenchus, Rotylenchulus, Belonolaimus, Heterodera, other cyst nematodes, Meloidogyne, Criconemoides, Hemicycliophora, Paratylenchus, Tylenchulus, Aphelenchoides, Bursaphelenchus, Rhadinaphelenchus, Longidorus, Xiphinema, Trichodorus, Paratrichodorus, Dirofiliaria, Onchocerca, Brugia, Acanthocheilonema, Aelurostrongylus, Anchlostoma, Angiostrongylus, Ascaris, Bunostomum, Capillaria, Chabertia, Cooperia, Crenosoma, Dictyocaulus, Dioctophyme, Dipetalonema, Dracunculus, Enterobius, Filaroides, Haemonchus, Lagochilascaris, Loa, Manseonella, Muellerius, Necator, Nematodirus, Oesophagostomum, Ostertagia, Parafilaria, Parascaris, Physaloptera, Protostrongylus, Setaria, Spirocerca, Stephanogilaria, Strongy hides, Strongylus, Thelazia, Toxascaris, Toxocara, Trichinella, Trichostrongylus, Trichuris, Uncinaria or Wuchereria. In an embodiment, the nematodes are of the genera Cooperia, Haemonchus, Caenorhabditis, Nippostrongyles, Dirofilaria, Onchocerca, Brugia, Acanthocheilonema, Dipetalonema, Loa, Mansonella, Parafilaria, Setaria, Stephanofilaria, Wucheria, Pratylenchus, Heterodera, Meloidogyne or Paratylenchus. In some embodiments the nemotodes are of the species Cooperia oncophora, Haemonchus contortus, Caenorhabditis elegans, Nippostrongyles brasiliensis, Ancylostoma caninum, Haemonchus contortus, T\acute{η}chinella spiralis, Trichurs muris, Dirofilaria immitis, Dirofilaria tenuis, Dirofilaria repens, Dirofilari ursi, Ascaris suum, Toxocara canis, Toxocara cati, Strongyloides ratti, Parastrongyloides trichosuri, Heterodera glycines, Globodera pallida, Meloidogyne javanica, Meloidogyne incognita, Meloidogyne arenaria, Radopholus similis, Longidorus elongatus, Meloidogyne hapla or Pratylenchus penetrans.

In an embodiment, the nematode infection is an infection of a nematode of a species selected from Cooperia oncophora, Haemonchus contortus, Dirofilaria, Nippostrongyles brasiliensis and Caenorhabditis elegans.

In an embodiment, the subject is selected from humans, mammals, birds, vertebrates, plants, seeds, and soil.

In an embodiment, the nematicidal compositions further comprise one or more agricultural excipients.

In an embodiment, the nematicidal compositions further comprise one or more agriculturally acceptable excipients.

For example, in an embodiment, the nematicidal compositions further comprises an aqueous surfactant. Examples of surfactants that can be used include, Span 20, Span 40, Span 80, Span 85, Tween 20, Tween 40, Tween 80, Tween 85, Triton X 100, Makon 10, Igepal CO 630, Brij 35, Brij 97, Tergitol TMN 6, Dowfax 3B2, Physan and Toximul TA 15, and mixtures therof.

In an embodiment, the nematicidal composition further comprises a permeation enhancer (e.g., cyclodextrin). In an embodiment, the nematicidal composition further comprises a co-solvent. Examples of co-solvents that can be used include ethyl lactate, methyl soyate/ethyl lactate co-solvent blends (e.g., Steposol), isopropanol, acetone, 1,2-propanediol, n-alkylpyrrolidones (e.g., the Agsolex series), a petroleum based-oil (e.g., aromatic 200) or a mineral oil (e.g., paraffin oil), or mixtures thereof. In an embodiment, the nematicidal composition further comprises another pesticide (e.g., nematicide, insecticide orfungicide) such as an avermectin (e.g., ivermectin), milbemycin, imidacloprid, aldicarb, oxamyl, fenamiphos, fosthiazate, metam sodium, etridiazole, penta-chloro-nitrobenzene (PCNB), flutolanil, metalaxyl, mefonoxam, and fosetyl-al, or mixtures thereof. Useful fungicides include, but are not limited to, silthiofam, fludioxonil, myclobutanil, azoxystrobin, chlorothalonil, propiconazole, tebuconazole and pyraclostrobin, or mixtures thereof. In an embodiment, the nematicidal composition may also comprise herbicides (e.g., trifloxysulfuron, glyphosate, halosulfuron) and other chemicals for disease control (e.g., chitosan).

In an embodiment, the application also includes a nematicidal feed for a non-human vertebrate including: (a) a feed; and (b) a nematicidal composition, of the application.

In an embodiment, the feed is selected from: soy, wheat, corn, sorghum, millet, alfalfa, clover, and rye, and mixtures therof. Also described are feeds that have been supplemented to include one or more of the compounds of the application. A nematicidal feed for a non-human vertebrate can comprise: (a) an animal feed; and (b) an effective amount of a nematicidal compound of the application.

In an embodiment, the pharmaceutical compositions further comprise one or more pharmaceutically acceptable excipients. Conventional procedures and ingredients for the selection and preparation of suitable pharmaceutical compositions are described, for example, in Remington's Pharmaceutical Sciences (2000-20th edition) and in The United States Pharmacopeia: The National Formulary (USP 24 NF19) published in 1999.

For example, the pharmaceutical forms suitable for injectable use include sterile aqueous solutions or dispersions and sterile powders for the extemporaneous preparation of sterile injectable solutions or dispersions. In all cases, the form is sterile and fluid to the extent that easy syringability exists.

In an embodiment, parenteral administration is by continuous infusion over a selected period of time. Solutions suitable for parenteral administration are prepared by known methods by a person skilled in the art. For example, the compounds of the application are prepared in water optionally mixed with a surfactant such as hydroxypropylcellulose. Dispersions are also prepared in glycerol, liquid polyethylene glycols, DMSO and mixtures thereof with or without alcohol, and in oils. Under ordinary conditions of storage and use, these preparations contain a preservative to prevent the growth of microorganisms.

Compositions for nasal administration are conveniently formulated as aerosols, drops, gels or powders. Aerosol formulations typically comprise a solution or fine suspension of the active substance in a physiologically acceptable aqueous or non-aqueous solvent and are usually presented in single or multidose quantities in sterile form in a sealed container, which take the form of a cartridge or refill for use with an atomising device. Alternatively, the sealed container is a unitary dispensing device such as a single dose nasal inhaler or an aerosol dispenser fitted with a metering valve, which is intended for disposal after use. Where the dosage form comprises an aerosol dispenser, it contains a propellant, which is, for example, a compressed gas such as compressed air or an organic propellant such as fluorochlorohydrocarbon. In an embodiment, the aerosol dosage forms take the form of a pump-atomizer.

Compositions suitable for buccal or sublingual administration include tablets, lozenges, and pastilles, wherein the active ingredient is formulated with a carrier such as sugar, acacia, tragacanth, gelatin and/or glycerine. Compositions for rectal administration are conveniently in the form of suppositories containing a conventional suppository base such as cocoa butter.

In another embodiment, compounds of the application are orally administered, for example, with an inert diluent or with an assimilable edible carrier, or they are enclosed in hard or soft shell gelatin capsules, or they are compressed into tablets, or they are incorporated directly with the food of a diet. For oral administration, the compounds of the application may be incorporated with excipients and used in the form of, for example, ingestible tablets, buccal tablets, troches, capsules, elixirs, suspensions, syrups, wafers, and the like. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., molecular sieving type particles, or, a fine hollow permeable fiber bundle, or chopped hollow permeable fibers, agglomerated or held in a fibrous packet. In an embodiment, timed-release compositions are, formulated, as liposomes or those wherein the active compound is protected with differentially degradable coatings, such as by microencapsulation, multiple coatings, etc. Liposome delivery systems include, for example, small unilamellar vesicles, large unilamellar vesicles and multilamellar vesicles. In an embodiment, liposomes are formed from a variety of lipids, such as cholesterol, stearylamine or phosphatidylcholines.

It is also possible to freeze-dry the compounds of the application and use the lyophilizate obtained, for example, for the preparation of products for injection.

The compounds of the application are either commercially available or may be prepared using methods known in the art.

The formation of a desired compound salt is achieved using standard techniques. For example, the neutral compound is treated with an acid or base in a suitable solvent and the formed salt is isolated by filtration, extraction or any other suitable method.

The formation of solvates will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art.

The following non-limiting examples are illustrative of the present application. As is apparent to those skilled in the art, many of the details of the examples may be changed while still practicing the methods, compositions and kits described herein.

### IV. Novel Compounds of the Application

Certain compounds of Formula (IV) are novel and therefore the present application includes these compounds and compositions comprising these compounds and uses thereof. Accordingly, the present application includes

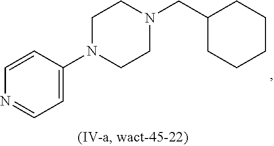 [SMILES: c1cc(N2CCN(CC3CCCCC3)CC2)ccn1]

or a pharmaceutically acceptable salt and/or solvate thereof.

The present application also includes a pharmaceutical composition comprising a compound of Formula IV-a or a pharmaceutically acceptable salt and/or solvate thereof, and a pharmaceutical acceptable carrier.

The present application also includes a nematicidal composition comprising a compound of Formula IV-a or a salt and/or solvate thereof, and a carrier.

### V. Methods of Preparation

The compounds of the application are either commercially available or may be prepared using methods known in the art. For example, in an embodiment, compounds of Formula (I), are prepared as shown in Scheme I:

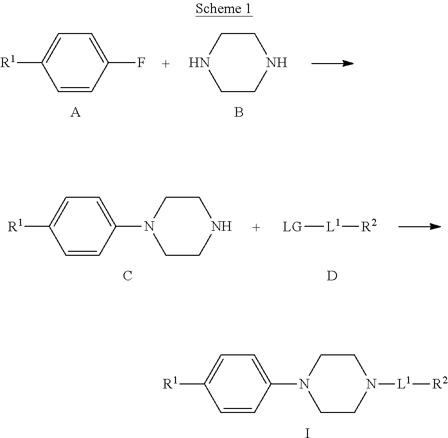

Therefore compounds of Formula (A), wherein R¹ is as defined in Formula (I), are reacted with excess amounts of piperazine (B) in the presence of an inorganic base at elevated temperatures to provide compounds of Formula C. Compounds of Formula C are then reacted with compounds of Formula D, wherein LG is a leaving group and L¹ and R² are as defined in Formula (I), in the presence of an organic base, at elevated temperatures, to provide compounds of Formula (I).

Compounds of Formula (II), Formula (III), Formula (IV), Formula (V), or Formula (VI) are also prepared as shown in Scheme I using a suitable corresponding compound of Formula A, C and D.

The formation of a desired compound salt is achieved using standard techniques. For example, the neutral compound is treated with an acid in a suitable solvent and the formed salt is isolated by filtration, extraction or any other suitable method.

The formation of solvates will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art.

The following non-limiting examples are illustrative of the present application. As is apparent to those skilled in the art, many of the details of the examples may be changed while still practicing the methods, compositions and kits described herein.

### EXAMPLES

### Example 1: Preparation of Compounds

### Materials and Methods

Unless otherwise stated, all reactions were carried out under an atmosphere of dry argon, using glassware that was either oven (120° C.) or flame-dried. Work-up and isolation of compounds was performed using standard benchtop techniques. All commercial reagents were purchased from chemical suppliers (Sigma-Aldrich, Combi-Blocks, or Alfa Aesar) and used without further purification. Dry solvents were obtained using standard procedures (dichloromethane and acetonitrile were distilled over calcium hydride). Reactions were monitored using thin-layer chromatography (TLC) on EMD Silica Gel 60 F254 plates. Visualization was performed under UV light (254 nm) or using potassium permanganate (KMnO₄) stain. Flash column chromatography was performed on Siliaflash P60 40-63 μm silica gel purchased from Silicycle.

NMR characterization data was obtained at 293K on a Varian Mercury 300 MHz, Varian Mercury 400 MHz, Bruker Advance III 400 MHz, Agilent DD2 500 MHz equipped with a 5 mm Xses cold probe or Agilent DD2 600 MHz. ¹H spectra were referenced to the residual solvent signal (CDCl₃=7.26 ppm, DMSO-d₆=2.50 ppm). ¹³C{¹H} spectra were referenced to the residual solvent signal (CDCl₃=77.16 ppm, DMSO-d_{6=39.52} ppm). Data for ¹H NMR are reported as follows: chemical shift (b ppm), multiplicity (s=singlet, d=doublet, t=triplet, q=quartet, m=multiplet), coupling constant (Hz), integration. NMR spectra were recorded at the University of Toronto Department of Chemistry NMR facility (http://www.chem.utoronto.ca/facilities/nmr/nmr.html).

Infrared spectra were recorded on a Perkin-Elmer Spectrum 100 instrument equipped with a single-bounce diamond/ZnSe ATR accessory in the solid state and are reported in wavenumber (cm⁻¹) units.

High resolution mass spectra (HRMS) were recorded at the Advanced Instrumentation for Molecular Structure (AIMS) in the Department of Chemistry at the University of Toronto (https://www.chem.utoronto.ca/chemistry/AIMS.php).

High-performance liquid chromatography (HPLC) analyses were performed on an Agilent 1100 series instrument containing a Phenomenex XDB-C18 column (1.8 μm, 50×4.6 mm) and run at room temperature. A linear gradient starting from 5% acetonitrile and 95% water (0.1% formic acid) to 95% acetonitrile and 5% water (0.1% formic acid) over 4 minutes followed by elution for 5 minutes at 95% acetonitrile and 5% water (0.1% formic acid) was used. The flow rate was set to 1.0 mL/min, with UV detection at 254 and 280 nm.

### General Exemplary Procedure A

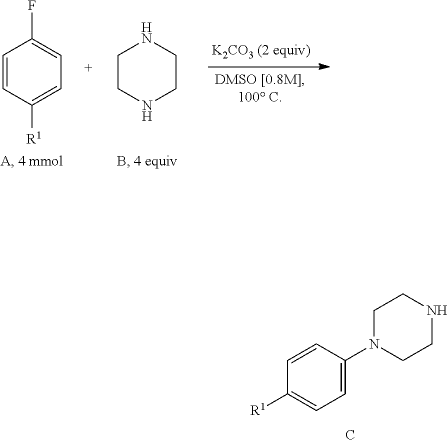

The procedure was modified from literature (Sokoloff, P.; Pilon, C.; Mann, A.; Schoenfelder, A.; Garrido, F. 3,4-dihydro-2-naphthamide derivatives as selective dopamine D3 ligands. European Patent Application 05290156.8, Jul. 26, 2006). To a round-bottom flask at room temperature were added piperazine (1.38 g, 16 mmol, 4.0 equiv), potassium carbonate (1.11 g, 8 mmol, 2 equiv), dimethyl sulfoxide (4.7 mL) then the corresponding fluorobenzene derivative (4 mmol, 1 equiv) and the mixture was stirred at 100° C. Once the reaction was complete as indicated by TLC (approximately 22 h), the reaction mixture was diluted with ethyl acetate, transferred to a separatory funnel and washed with water (3 times) then saturated sodium chloride. The organic phase was dried with Na₂SO₄, filtered, then concentrated on a rotary evaporator and the substituted 1-phenylpiperazine (C) was obtained. The crude solid was used in the next step without further purification.

### General Procedure B

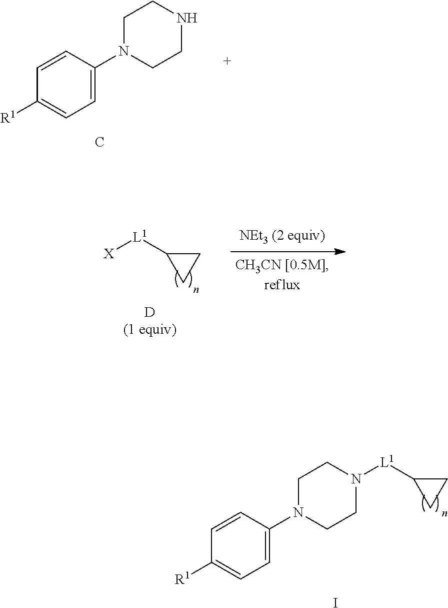

To a round-bottom flask were added C (0.5 mmol, 1.0 equiv), dry acetonitrile (1 mL), triethylamine (0.14 mL, 1 mmol, 2 equiv) then the alkyl halide wherein X is a halide (D, 0.5 mmol, 1.0 equiv) and the mixture was stirred at reflux until completion as indicated by TLC (approximately 24 h). The mixture was diluted with ethyl acetate, transferred to a separatory funnel and washed with water (3 times) then saturated sodium chloride. The organic phases were combined and dried with Na₂SO₄, filtered, and concentrated on a rotary evaporator. The residue was purified by column chromatography with the indicated eluent and the alkylated arylpiperazines (1) were obtained.

Preparation of Cyclopentylmethyl methylbenzenesulfonate

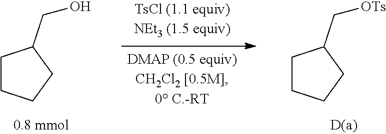 [SMILES: OCC1CCCC1 ; Cc1ccc(S(=O)(=O)OCC2CCCC2)cc1]

The procedure was modified from literature (Shi, W.; Nacev, B. A.; Aftab, B. T.; Head, S.; Rudin, C. M.; Liu, J. O. J. Med. Chem. 2011, 54, 7363). To a round-bottom flask were added cyclopentylmethanol (0.087 mL, 0.8 mmol, 1.0 equiv) and dry dichloromethane (1.6 mL) and the flask was submerged in an ice-water bath. Triethylamine (0.17 mL, 1.2 mmol, 1.5 equiv) was added to the flask followed by 4-(dimethylamino)pyridine (48.9 mg, 0.4 mmol, 0.5 equiv) then p-toluenesulfonyl chloride (168 mg, 0.88 mmol, 1.1 equiv) and the mixture was warmed to room temperature and stirred until completion as indicated by TLC (approximately 3 h). Dichloromethane and water were added, then the mixture was transferred to a separatory funnel and the layers were separated. The organic phase was washed with water (2 times) followed by saturated sodium chloride, then dried over Na₂SO₄, filtered, and concentrated on a rotary evaporator. The residue was purified by column chromatography (19/1 v/v pentanes/ethyl acetate) and cyclopentylmethyl 4-methylbenzenesulfonate (D(a), 59% yield) was obtained as a colourless oil. The spectral data was in accordance with literature.

### Example 2 Bioactivity Profile of Exemplary Compounds of Formula (I), Formula (II), Formula (III), Formula (IV), Formula (V), and Formula (VI)

Table 1 shows the bioactivity profile study conducted on exemplary compounds of Formula (I), Formula (II), Formula (III), Formula (IV), Formula (V) and Formula (VI), where data are the % of C. elegans L3 larvae demonstrating abnormal locomotory behaviour on solid agar (MYOB medium+E. coli OP50 food source) containing the indicated compound of the application at 3.75 uM, 15 uM or 60 uM (as noted). 20 animals were scored after either 1, 6 or 24 hours of exposure to drug (as indicated) per replicate. Abnormal locomotory phenotype is defined as animals demonstrating 1 or more of the following phenotypes: jerky locomotion, flaccid paralysis, coiling. Data are reported as the mean % of animals with abnormal phenotype over 4 independent replicates.

TABLE 1

| | % C. elegans L3 larvae | | | % C. elegans L3 larvae | | | % C. elegans L3 larvae | | |
|---|---|---|---|---|---|---|---|---|---|
| | any phene 1 h (conc.) | | | any phene 6 h (conc.) | | | any phene 24 h (conc.) | | |
| Compound I.D. | 3.75 | 15 | 60 | 3.75 | 15 | 60 | 3.75 | 15 | 60 |
| Control | 0 | 0 | 0 | 0 | 0 | 0 | 0 | 0 | 0 |
| I-a, wact-45-11 | 3.3 | 6.7 | 19.2 | 0.0 | 0.0 | 5.8 | 0.0 | 0.0 | 0.0 |
| II-a, wact-45-14 | 0.8 | 0.0 | 6.7 | 0.0 | 0.0 | 2.5 | 0.0 | 0.0 | 0.0 |
| II-b, wact-45-15 | 0.0 | 2.5 | 23.3 | 0.8 | 0.0 | 29.2 | 0.0 | 0.0 | 0.0 |
| III-a wact-45-16 | 0.8 | 0.8 | 4.2 | 0.8 | 5.8 | 24.2 | 0.0 | 0.0 | 0.0 |
| III-b, wact-45-17 | 0.0 | 0.0 | 9.2 | 0.0 | 0.0 | 8.3 | 0.0 | 0.0 | 0.0 |
| III-c, wact-45-18 | 0.0 | 10.0 | 27.5 | 0.0 | 0.0 | 15.0 | 0.0 | 0.0 | 0.0 |
| III-d, wact-45-20 | 0.8 | 14.2 | 30.8 | 0.0 | 1.7 | 17.5 | 0.0 | 0.0 | 0.0 |
| IV-a, wact-45-22 | 0.0 | 10.8 | 24.2 | 0.0 | 0.0 | 12.5 | 0.0 | 0.0 | 0.0 |
| V-a, wact-45-23 | 15.8 | 33.3 | 63.3 | 0.0 | 15.8 | 27.5 | 0.0 | 0.0 | 0.0 |
| V-b, wact-45-25 | 2.5 | 20.8 | 62.5 | 0.0 | 3.3 | 41.7 | 0.0 | 0.0 | 9.2 |
| VI-a, wact-45-41 | 58.3 | 98.3 | 100.0 | 1.7 | 74.2 | 100.0 | 0.0 | 20.0 | 100.0 |
| VI-c, wact-45-43 | 33.8 | 100.0 | 100.0 | 7.5 | 64.2 | 84.2 | 0.0 | 10.0 | 100.0 |
| VI-d, wact-45-44 | 80.0 | 100.0 | 100.0 | 11.7 | 59.2 | 100.0 | 0.0 | 0.0 | 66.7 |
| VI-e, wact-45-45 | 36.7 | 96.7 | 100.0 | 5.8 | 46.7 | 100.0 | 0.0 | 5.0 | 99.2 |
| VI-f, wact-45-47 | 34.2 | 81.7 | 100.0 | 0.0 | 83.2 | 100.0 | 0.0 | 52.5 | 92.5 |
| VI-g, wact-45-49 | 9.2 | 65.0 | 94.2 | 0.0 | 21.7 | 54.2 | 0.0 | 0.0 | 1.7 |
| VI-h, wact-45-52 | 64.2 | 100.0 | 100.0 | 0.0 | 37.5 | 100.0 | 0.0 | 25.0 | 62.5 |
| VI-i, wact-45-55 | 26.7 | 70.0 | 96.7 | 0.0 | 0.0 | 58.3 | 0.0 | 0.0 | 5.0 |
| VI-j, wact-45-56 | 25.8 | 69.2 | 100.0 | 0.8 | 12.5 | 41.7 | 0.0 | 0.0 | 9.2 |
| IV-b, wact-45-60 | 4.2 | 21.7 | 30.0 | 0.0 | 0.8 | 4.2 | 0.0 | 0.0 | 1.7 |
| VI-k, wact-45-63 | 18.3 | 94.2 | 95.0 | 0.8 | 30.8 | 70.0 | 0.0 | 8.3 | 80.8 |
| vesamicol | 0.0 | 0.0 | 11.7 | 0.0 | 0.8 | 1.7 | 0.0 | 0.0 | 0.0 |

### Example 3 Further Bioactivity Profile of Exemplary Compounds of Formula (I), Formula (II), Formula (III), Formula (IV), Formula (V), and Formula (VI)

Table 2 shows further bioactivity profile studies conducted on exemplary compounds of Formula (I), Formula (II), Formula (III), Formula (IV), Formula (V) and Formula (VI), Pristionchus pacificus data are the determined EC50s of young adult animals demonstrating abnormal locomotory behaviour on solid agar (MYOB medium+E. coli OP50 food source) containing the indicated compound of the application (as noted). 20 animals were scored after 1 hour of exposure to drug (as indicated) per replicate. Abnormal locomotory phenotype is defined as animals demonstrating 1 or more of the following phenotypes: jerky locomotion, flaccid paralysis, coiling. Data are reported as the EC50 of animals with abnormal phenotype calculated over 3 independent replicates testing at 3.75 μM, 15 μM and 60 μM in technical duplicate. A reported EC50 as ‘>60 μM’ signifies ≤550% effect was observed at the 60 μM test concentration; NA represents ‘not active’ and signifies no phenotype was observed at the 60 μM test concentration. Experiments on D. immitis microfilariae and larval stage 3 (L3) worms were performed in the laboratories of Bayer Animal Health GmbH (Monheim, Germany) in accordance with the local Animal Care and Use Committee and governmental authorities (LANUV #200/A176 and #200/A154). For microfilariae immobility assays, approximately 250 freshly purified microfilariae were cultured in single wells of a 96-well microtiter plate containing supplemented RPMI 1640 medium. Compounds were added in the following concentrations: 50 μM, 10 μM, 2 μM, 0.4 μM, 0.08 μM, 0.016 μM and 0.0032 μM. Microfilariae exposed to medium substituted with 1% DMSO were used as negative controls. Motility of microfilariae was evaluated after 72 h of drug exposure using an image-based approach—Dirolmager, developed by Bayer Technology Services. Data are reported as the EC50 (μM) calculated from the tested concentration series. For L3 development assays, freshly isolated L3s were cultured in wells of a 96-well microtiter plate with 10 L3 per well. All wells contained supplemented RPMI 1640 medium and test compound at one of the following concentrations: 10 μM, 2 μM, 0.4 μM, 0.08 μM, 0.016 μM and 0.0032 μM. L3s exposed to DMSO only (1%) were used as negative controls. All drug concentrations were tested in duplicate and drug effects were evaluated after 72 h of incubation. Motility was scored for each individual worm. Each motile worm, independently of the degree of motility, reduced the drug activity by 5%, as two cavities with 10 worms each (20 worms in total=100% activity if all of them are completely paralyzed) were tested per indicated compound of the application (as noted). Data were only considered as valid if at least 90% worms in the negative control group remained as motile as observed at the beginning of the experiment. Data are reported as the EC50 (μM) calculated from the tested concentration series. Strongyloides ratti lethality data are the measurement of the % of adult or larval stage L3 animals (as indicated) that respond to an 80° C. water stimulus after 72 h of incubation in wells containing the indicated compound of the application (as noted). Data are the mean measurement of 30-40 larvae incubated for 72 h over 1 replicate. Heligmosomoides polygyrus lethality data are determined by the measurement of the % of larval stage 3 (L3) worms that respond to an 80° C. water stimulus hot water stimulus after 72 h of incubation in wells containing the indicated compound of the application (as noted). Data are the mean measurement of 30-40 larvae incubated in a dark box at room temperature for 72 h over one replicate. Relative binding affinity of the indicated compound of the application (as noted) is reported as the inhibition constant (Ki) determined from competition binding assays measuring the displacement of [³H]vesamicol bound to rat VAChT expressed in P12 cells. Danio rerio (zebrafish) phenotype data are the measurement of any abnormal developmental phenotypes of 1 day post fertilization embryos through 48 h of development. Abnormal developmental phenotypes are defined as any gross morphological differences in development compared to parallel controls, lethality or measured significant reduction in motor response from control using an automated locomotion tracker. Data are EC50s calculated from the mean of three replicates of six D. rerio embryos per replicate measured at 15 μM and 3.75 μM with select compounds tested at 60 μM and 30 μM. >15 μM or >60 μM indicate that a 50% effect was not observed at the highest concentration tested.

TABLE 2

| | | D. immitis | | | | |
|---|---|---|---|---|---|---|
| | P. pacificus; | microfilariae | | | | D. rerio |
| | young adult 1 h | immobility//L3 | S. ratti | H. Polygyrus | | (zebrafish) any |
| | any phene at 1 h | development | L3 % lethality | % lethality L3 | ratVAChT | phenotype |
| Compound | (EC50 (μM)) | EC50 (μM) | 25 μM | 25 μM | Ki (nM) | EC50 (μM) |
| control | — | NA | NA | NA | | — |
| I-a, wact-45-11 | >60 | NA | | | | >15 |
| II-a, wact-45-14 | >60 | NA | | | | — |
| II-b, wact-45-15 | >60 | NA | | | | — |
| III-a wact-45-16 | >60 | NA//>50 | | | | >15 |
| III-b, wact-45-17 | >60 | NA | | | | 7.8 |
| III-c, wact-45-18 | >60 | NA | | | | >15 |
| III-d, wact-45-20 | >60 | NA | | | | >15 |
| IV-a, wact-45-22 | | NA | | | | >15 |
| V-a, wact-45-23 | >60 | NA//15.0 | | | | 5.1 |
| V-b, wact-45-25 | >60 | 5.1//14.0 | | | | 5.1 |
| VI-a, wact-45-41 | 10.7 | NA//>50 | 28.1 | 31.6 | 411 | >15 |
| VI-c, wact-45-43 | — | NA//>50 | 0 | 7.6 | 159 | 16.9 |
| VI-d, wact-45-44 | 2.5 | NA | 17.9 | 56.5 | | 15.0 |
| VI-e, wact-45-45 | 5.7 | NA | 31.6 | 0 | 208 | 32.5 |
| VI-f, wact-45-47 | 2.8 | NA | 0 | 0 | | 7.8 |
| VI-g, wact-45-49 | — | NA | 0 | 11.6 | | 29.3 |
| VI-h, wact-45-52 | 21.7 | NA | 0 | 0 | | 7.8 |
| VI-i, wact-45-55 | 49.6 | NA//>50 | 0 | 11.2 | | 4.0 |
| VI-j, wact-45-56 | 41.5 | NA | 66.7 | 35.4 | | 21.1 |
| IV-b, wact-45-60 | NA | NA | | | | >15 |
| VI-k, wact-45-63 | >60 | NA | 0 | 34.1 | 6680 | >60 |
| vesamicol | >60 | NA | 17.3 | 0 | 30.3 | 21.1 |

While the present application has been described with reference to what are presently considered to be the preferred examples, it is to be understood that the application is not limited to the disclosed examples. To the contrary, the present application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

1. A method of treating or preventing a nematode infection or of treating or preventing a disease, disorder or condition arising from a nematode infection comprising administering, to a subject in need thereof, an effective amount of a compound of Formula (VI) 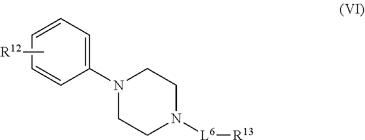
and/or a pharmaceutically acceptable salt and/or solvate thereof wherein:
R¹² is C₁₋₄alkyl, C₁₋₄fluoroalkyl, NO₂, Cl, F or CN;
L⁶ is a direct bond or C₁₋₄alkylene; and
R¹³ is C₃₋₈cycloalkyl or C₃₋₈cycloalkenyl, each of which is unsubstituted or substituted with one or two substituents independently selected from OH, F, Cl, Br, CN, C₁₋₄alkyl, C₁₋₄fluoroalkyl, OC₁₋₄alkyl, OC₁₋₄fluoroalkyl, and OC₁₋₄fluoroalkyl,
provided when R¹² is NO₂ or CN and is in a position para to the piperazine on the phenyl ring, then R¹³ is not C₃₋₈cycloalkyl or C₃₋₈cycloalkenyl each of which is unsubstituted, or C₃₋₈cycloalkyl or C₃₋₈cycloalkenyl each of which is substituted with one or two substituents independently selected from C₁₋₄alkyl and C₁₋₄fluoroalkyl; or
an effective amount of a compound of Formula (II) 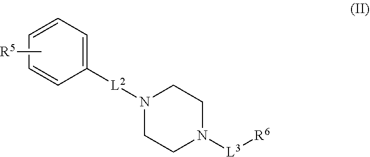
and/or a pharmaceutically acceptable salt and/or solvate thereof wherein:
R⁵ is C₁₋₄alkyl, C₁₋₄fluoroalkyl, NO₂, Cl, F or CN;
L² is C₁₋₄alkylene;
L³ is a direct bond or C₁₋₄alkylene; and
R⁶ is C₃₋₈cycloalkyl or C₃₋₈cycloalkenyl, each of which is unsubstituted or substituted with one or two substituents independently selected from OH, F, Cl, Br, CN, C₁₋₄alkyl, C₁₋₄fluoroalkyl, OC₁₋₄alkyl and OC₁₋₄fluoroalkyl; or
an effective amount of a compound of Formula (III) 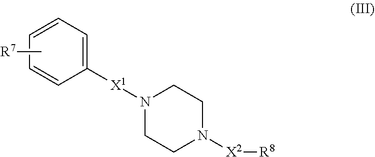
and/or a pharmaceutically acceptable salt and/or solvate thereof wherein:
R⁷ is H, C₁₋₄alkyl, C₁₋₄fluoroalkyl, NO₂, Cl, F or CN;
one of X¹ or X² is C═O and the other is a direct bond; and
R⁸ is phenyl, C₃₋₈cycloalkyl or C₃₋₈cycloalkenyl, each of which is unsubstituted or substituted with one or two substituents independently selected from OH, F, Cl, Br, CN, C₁₋₄alkyl, C₁₋₄fluoroalkyl, OC₁₋₄alkyl and OC₁₋₄fluoroalkyl; or
an effective amount of a compound of Formula (IV) 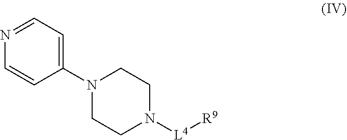
and/or a pharmaceutically acceptable salt and/or solvate thereof wherein:
L⁴ is a direct bond or C₁₋₄alkylene; and
R⁹ is C₃₋₈cycloalkyl or C₃₋₈cycloalkenyl, each of which is unsubstituted or substituted with one or two substituents independently selected from OH, F, Cl, Br, CN, C₁₋₄alkyl, C₁₋₄fluoroalkyl, OC₁₋₄alkyl and OC₁₋₄fluoroalkyl; or
an effective amount of a compound of Formula (V) 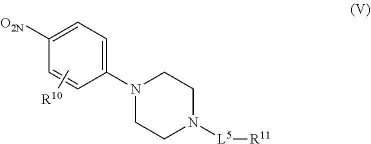
and/or a pharmaceutically acceptable salt and/or solvate thereof wherein:
R¹⁰ is C₁₋₄alkyl, C₁₋₄fluoroalkyl, Cl, F or CN;
L⁵ is a direct bond or C₁₋₄alkylene; and
R¹¹ is C₃₋₈cycloalkyl or C₃₋₈cycloalkenyl, each of which is unsubstituted or substituted with one or two substituents independently selected from OH, F, Cl, Br, CN, C₁₋₄alkyl, C₁₋₄fluoroalkyl, OC₁₋₄alkyl and OC₁₋₄fluoroalkyl; or
an effective amount of a compound of Formula (I) 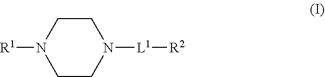
and/or a pharmaceutically acceptable salt and/or solvate wherein:
R¹ is C₁₋₄alkyl;
R² is C₃₋₈cycloalkyl or C₃₋₈cycloalkenyl, each of which is unsubstituted or substituted with one or two substituents independently selected from OH, F, Cl, Br, CN, C₁₋₄alkyl, C₁₋₄fluoroalkyl, OC₁₋₄alkyl and OC₁₋₄fluoroalkyl; and
L¹ is a direct bond or C₁₋₄alkylene.

2. The method of claim 1, wherein R¹² is C₁₋₂alkyl, C₁₋₂fluoroalkyl, NO₂, Cl, F or CN.

3. The method of claim 1, wherein L⁶ is a direct bond.

4. The method of claim 1, wherein L⁶ is C₁₋₄alkylene.

5. The method of claims 1 to 4, wherein R¹³ is C₃₋₈cycloalkenyl.

6. (canceled)

7. The method of claim 5, wherein the C₃₋₈cycloalkenyl is a bicyclic cycloalkenyl.

8.-9. (canceled)

10. The method of claim 1, wherein R¹³ is C₃₋₈cycloalkyl.

11.-15. (canceled)

16. The method of claim 1, wherein the compound of Formula (VI) is selected from 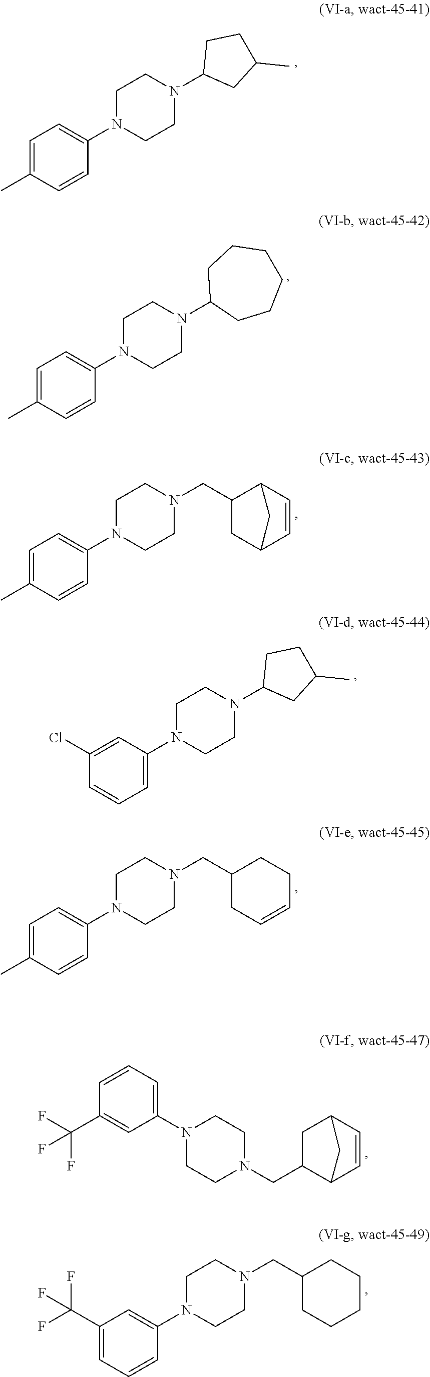 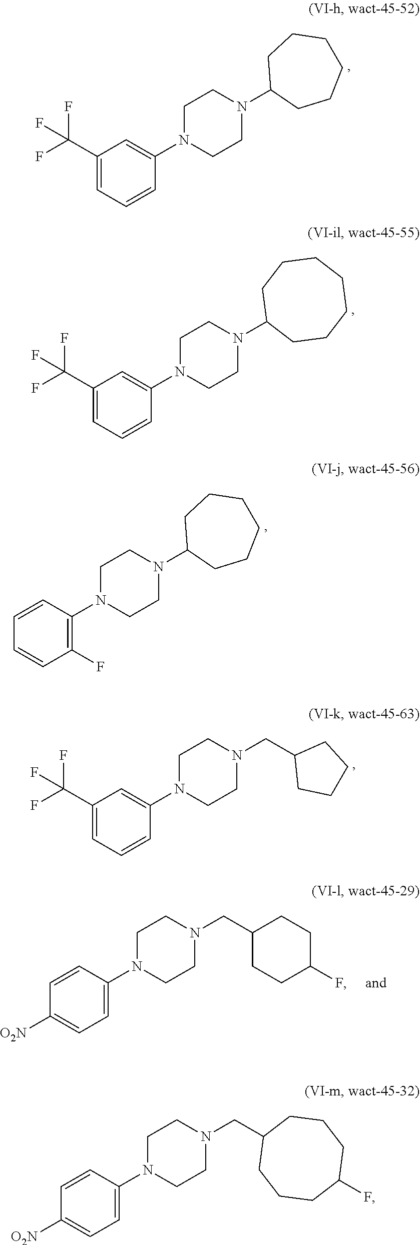
or a pharmaceutically acceptable salt and/or solvate thereof:
the compound of Formula (II) is selected from 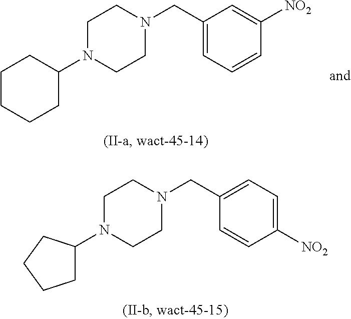 [SMILES: O=[N+]([O-])c1cccc(CN2CCN(C3CCCCC3)CC2)c1 ; O=[N+]([O-])c1ccc(CN2CCN(C3CCCC3)CC2)cc1]
or a pharmaceutically acceptable salt and/or solvate thereof:
the compound of Formula (III) is selected from 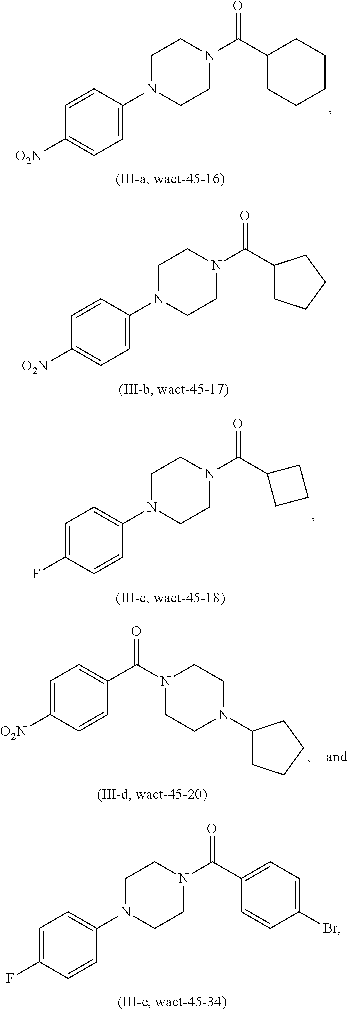 [SMILES: O=C(C1CCCCC1)N1CCN(c2ccc([N+](=O)[O-])cc2)CC1 ; O=C(C1CCCC1)N1CCN(c2ccc([N+](=O)[O-])cc2)CC1 ; O=C(C1CCC1)N1CCN(c2ccc(F)cc2)CC1 ; O=C(c1ccc([N+](=O)[O-])cc1)N1CCN(C2CCCC2)CC1 ; O=C(c1ccc(Br)cc1)N1CCN(c2ccc(F)cc2)CC1]
or a pharmaceutically acceptable salt and/or solvate thereof:
the compound of Formula (IV) is selected from 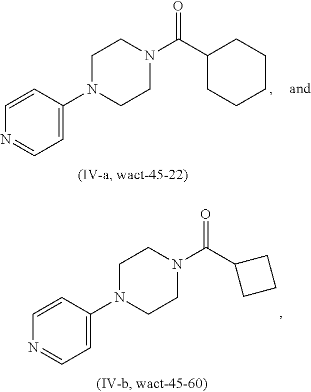 [SMILES: O=C(C1CCCCC1)N1CCN(c2ccncc2)CC1 ; O=C(C1CCC1)N1CCN(c2ccncc2)CC1]
or a pharmaceutically acceptable salt and/or solvate thereof:
the compound of Formula (V) is selected from 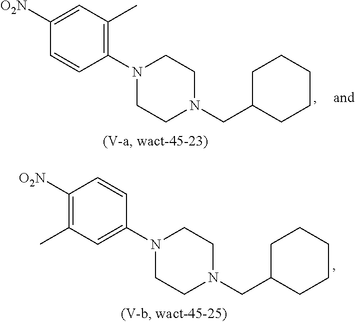 [SMILES: Cc1cc([N+](=O)[O-])ccc1N1CCN(CC2CCCCC2)CC1 ; Cc1cc(N2CCN(CC3CCCCC3)CC2)ccc1[N+](=O)[O-]]
or a pharmaceutically acceptable salt and/or solvate thereof; and
the compound of Formula (I) is 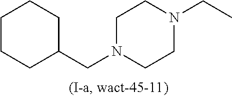
or a pharmaceutically acceptable salt and/or solvate thereof.

17. (canceled)

18. The method of claim 1, wherein R⁵ is C₁₋₂alkyl, C₁₋₂fluoroalkyl, NO₂, Cl, F or CN.

19. The method of claim 1, wherein L² is C₁₋₃alkylene.

20. The method of claim 1, wherein L³ is a direct bond.

21. (canceled)

22. The method of claim 1, wherein R⁶ is C₃₋₈cycloalkenyl.

23. (canceled)

24. The method of claim 22, wherein the C₃₋₈cycloalkenyl is a bicyclic cycloalkenyl.

25.-26. (canceled)

27. The method of claim 1, wherein R⁶ is C₃₋₈cycloalkyl.

28.-92. (canceled)

93. The method claim 1, wherein the nematode infection is an infection of a nematode of the following genera: Caenorhabditis, Nippostrongyles, Anguina, Ditylenchus, Tylenchorhynchus, Pratylenchus, Radopholus, Hirschmanniella, Nacobbus, Hoplolaimus, Scutellonema, Rotylenchus, Helicotylenchus, Rotylenchulus, Belonolaimus, Heterodera, other cyst nematodes, Meloidogyne, Criconemoides, Hemicycliophora, Paratylenchus, Tylenchulus, Aphelenchoides, Bursaphelenchus, Rhadinaphelenchus, Longidorus, Xiphinema, Trichodorus, Paratrichodorus, Dirofiliaria, Onchocerca, Brugia, Acanthocheilonema, Aelurostrongylus, Anchlostoma, Angiostrongylus, Ascaris, Bunostomum, Capillaria, Chabertia, Cooperia, Crenosoma, Dictyocaulus, Dioctophyme, Dipetalonema, Dracunculus, Enterobius, Filaroides, Haemonchus, Lagochilascaris, Loa, Manseonella, Muellerius, Necator, Nematodirus, Oesophagostomum, Ostertagia, Parafilaria, Parascaris, Physaloptera, Protostrongylus, Setaria, Spirocerca, Stephanogilaria, Strongy hides, Strongylus, Thelazia, Toxascaris, Toxocara, Trichinella, Trichostrongylus, Trichuris, Uncinaria or Wuchereria. The method of claim, wherein the nematodes are of the genera Cooperia, Haemonchus, Caenorhabditis, Nippostrongyles, Dirofilaria, Onchocerca, Brugia, Acanthocheilonema, Dipetalonema, Loa, Mansonella, Parafilaria, Setaria, Stephanofilaria, Wucheria, Pratylenchus, Heterodera, Meloidogyne or Paratylenchus. In some embodiments the nemotodes are of the species Cooperia oncophora, Haemonchus contortus, Caenorhabditis elegans, Nippostrongyles brasiliensis, Ancylostoma caninum, Haemonchus contortus, T\acute{η}chinella spiralis, Trichurs muris, Dirofilaria immitis, Dirofilaria tenuis, Dirofilaria repens, Dirofilari ursi, Ascaris suum, Toxocara canis, Toxocara cati, Strongyloides ratti, Parastrongyloides trichosuri, Heterodera glycines, Globodera pallida, Meloidogyne javanica, Meloidogyne incognita, Meloidogyne arenaria, Radopholus similis, Longidorus elongatus, Meloidogyne hapla or Pratylenchus penetrans.

94. The method of claim 1, wherein the nematode infection is an infection of a nematode of a species selected from Cooperia oncophora, Haemonchus contortus, Dirofilaria, Nippostrongyles brasiliensis and Caenorhabditis elegans.

95. The method claim 1, wherein the subject is selected from humans, mammals, birds, vertebrates, plants, seeds, and soil.

96. A pharmaceutical composition comprising a pharmaceutically acceptable carrier and a compound of Formula (I) as defined in claim 1, and/or a pharmaceutically acceptable salt and/or solvate thereof, wherein the compound of Formula (I) is present in an amount effective to treat a nematode infection in a subject in need thereof,
a compound of Formula (II) as defined in claim 1, and/or a pharmaceutically acceptable salt and/or solvate thereof, wherein the compound of Formula (II) is present in an amount effective to treat a nematode infection in a subject in need thereof;
a compound of Formula (III) as defined in claim 1, and/or a pharmaceutically acceptable salt and/or solvate thereof, wherein the compound of Formula (III) is present in an amount effective to treat a nematode infection in a subject in need thereof;
a compound of Formula (V) as defined in claim 1, and/or a pharmaceutically acceptable salt and/or solvate thereof, wherein the compound of Formula (V) is present in an amount effective to treat a nematode infection in a subject in need thereof;
a compound of Formula (VI) as defined in claim 1, and/or a pharmaceutically acceptable salt and/or solvate thereof, wherein the compound of Formula (VI) is present in an amount effective to treat a nematode infection in a subject in need thereof;
a compound of Formula (IV) as defined in claim 1, and/or a pharmaceutically acceptable salt and/or solvate thereof, wherein the compound of Formula (IV) is present in an amount effective to treat a nematode infection in a subject in need thereof.

97.-101. (canceled)

102. A compound that is 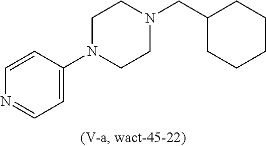 [SMILES: c1cc(N2CCN(CC3CCCCC3)CC2)ccn1]
or a pharmaceutically acceptable salt and/or solvate thereof.

103. A pharmaceutical composition comprising the compound of claim 102 or pharmaceutically acceptable salt and/or solvate thereof, and a pharmaceutical acceptable carrier.
